(19) United States
(12) Patent Application Publication
(10) Pub. No.: US 2020/0038641 A1
(43) Pub. Date: Feb. 6, 2020

(54) DRUG DELIVERY DEVICE WITH REMOVABLE PODS AND RELATED PODS, METHODS AND SYSTEMS

(71) Applicant: AURITEC PHARMACEUTICALS, Pasadena, CA (US)

(72) Inventors: Thomas J. SMITH, PASADENA, CA (US); Sarjan SHAH, PASADENA, CA (US)

(21) Appl. No.: 16/598,862

(22) Filed: Oct. 10, 2019

Related U.S. Application Data
(63) Continuation of application No. PCT/US2018/028425, filed on Apr. 19, 2018.
(60) Provisional application No. 62/487,415, filed on Apr. 19, 2017.

(51) Int. Cl. A61M 31/00; A61K 9/00; A61K 9/28; A61K 31/4439; A61K 38/08; A61P 31/22; A61P 5/08; A61J 3/06

(52) U.S. Cl. CPC A61M 31/002; A61K 9/0036; A61K 9/284; A61K 31/4439; A61M 2210/1475; A61P 31/22; A61P 5/08; A61J 3/06; A61K 38/08

(57) ABSTRACT

A drug delivery device with removable pods is described and related pods methods and systems. The drug delivery device comprises a plurality of receptacles, each receptacle configured to accept a corresponding drug pod, wherein each receptacle comprises: a recess configured to accept a protrusion in the corresponding drug pod, or a protrusion configured to insert in a recess in the corresponding drug pod. The corresponding drug pod comprises a shell, made of an impermeable polymer, having a first base comprising an opening configured to allow delivery of a drug, a second base, a lateral surface, a protrusion attached to, and extending away from, the lateral surface, or a recess in the lateral surface.

### CROSS REFERENCE TO RELATED APPLICATIONS

The present application is a continuation of International Application No. PCT/US2018/028425 filed on Apr. 19, 2018 which claims priority to U.S. Provisional Application No. 62/487,415, entitled “Drug Delivery Device with Removable Pods” filed on Apr. 19, 2017 with docket number P2026-USP, the contents of all of which are incorporated herein by reference in their entireties.

### STATEMENT OF GOVERNMENT GRANT

This invention was made with government support under Grant No. R44MH110161 awarded by the National Institutes of Health. The government has certain rights in the invention.

### FIELD

The present disclosure relates to drug delivery devices and drug release performed using the devices and in particular to drug delivery device with removable pods and related pods, compositions, methods, and systems.

### BACKGROUND

Over the last few decades, various drug delivery devices have been developed for the delivery of drugs both topically and systemically to vaginal, rectal or other tissues.

Despite the efforts made to develop new technology in this field, development of devices allowing controlled release of a drug and/or delivery of multiple drugs and/or delivery is still challenging with particular reference to devices such as intravaginal rings, sub-dermal implants, and pessaries/suppositories, as well as other devices for topical or systematic administration of a drug.

### SUMMARY

Provided herein are, devices and related pods, compositions methods and systems which allow in several embodiments controlled release of a drug comprised within the pods and/or delivery of a plurality of drugs to be possibly performed in a combination customizable for patient based on the patient need.

In a first aspect of the disclosure, a drug pod is described comprising: a shell, made of an impermeable polymer, having a first base comprising an opening configured to allow delivery of a drug, a second base, a lateral surface, a protrusion attached to, and extending away from, the lateral surface, or a recess in the lateral surface. In some embodiments, the drug pod optionally further comprises a drug core within the shell and/or a semi-permeable and/or permeable polymer layer on the first base, the semi-permeable and/or permeable polymer layer covering the opening in the first base. In some embodiments, the drug pod can further comprise a wick. In some embodiments, the semipermeable polymer and/or permeable layer can be crosslinked.

In a second aspect of the disclosure, a blank carrier ring device is described, comprising a ring, the ring having a plurality of cylindrical openings, each opening configured to accept a corresponding drug pod, wherein each opening comprises: a recess configured to accept a protrusion in the corresponding drug pod, or a protrusion configured to insert in a recess in the corresponding drug pod.

In a third aspect of the disclosure, a drug delivery system is described, the drug delivery system comprising: a blank carrier ring device and at least one drug pod. In the drug delivery system the blank carrier ring device comprises a ring, the ring having a plurality of receptacles, each receptacle configured to accept a corresponding drug pod, wherein each receptacle comprises: a recess configured to accept a protrusion in the corresponding drug pod, or a protrusion configured to insert in a recess in the corresponding drug pod. In the drug delivery system the at least one drug pod, configured to be inserted in a receptacle of the plurality of receptacles, the at least one drug pod comprising: a shell, made of an impermeable polymer, having a first base comprising an opening configured to allow delivery of a drug, a second base, a lateral surface, a protrusion attached to, and extending away from, the lateral surface, and optionally a semi-permeable polymer layer on the first base, the semi-permeable polymer layer covering the opening in the first base, and a drug core within the shell.

In a fourth aspect of the disclosure, a method is described comprising fabricating a pill comprising an active pharmaceutical compound with or without an excipient; inserting the pill in a shell, made of an impermeable polymer, the shell having a first base comprising an opening configured to allow delivery of a drug, a second empty base, a lateral surface, and optionally a semi-permeable polymer layer on the first base, the semi-permeable polymer layer covering the opening in the first base, the inserting being carried out through the second empty base; sealing the second empty base; and sealing the opening with a second semi-permeable polymer, thereby obtaining a sealed shell containing a pill, wherein the shell comprises: a protrusion attached to, and extending away from, the lateral surface, or a recess in the lateral surface. In some embodiments the method can further comprise cross-linking the semipermeable polymer within the shell containing the pill.

In a fifth aspect of the disclosure, a method for contraception and/or for treating and/or preventing one or more diseases in an individual is described. The method comprises providing a drug delivery system herein described comprising a plurality of drug cores within a plurality of drug pods, each drug core of the plurality of drug cores within a corresponding drug pod of the plurality of drug pods, each drug core of the plurality of drug cores and corresponding pod selected to provide the individual with one or more target active pharmaceutical ingredient at one or more effective target concentrations. The method further comprises administering the drug delivery system to the individual to deliver the one or more target active pharmaceutical ingredients to the individual. In some embodiments, the disease can be selected from the group consisting of vaginal diseases, uterine diseases, pelvic diseases, rectal diseases, eye diseases, ear diseases, sinus diseases, nasal diseases, prostatic diseases, and bladder disease. In various embodiments, one or more diseases can be treated while one ore more diseases can be prevented by administering a single drug delivery system configured in accordance with the present disclosure.

Accordingly in some embodiments a drug delivery system is herein described for use in contraception and/or for treatment and/or prevention of one or more diseases in an individual. In particular in some embodiments of the drug delivery system herein described one or more target drugs can be delivered to the individual at one or more effective target concentrations by selecting a combination of drug cores and corresponding drug pods which are configured to provide one or more target drugs to the individual at the one or more target concentrations effective to treat or prevent the one or more diseases in the individual.

Drug delivery devices and related components, compositions methods and systems herein described allow in several embodiments, to provide a patient with a combination of a plurality of drugs to be the delivered topically or systemically within a same device.

Drug delivery devices and related components, compositions methods and systems herein described allow in several embodiments, to provide controlled release of one more drugs from the device through a combination of pods selected to provide an individual with the one or more drugs at one or more selected effective target concentration that can be placed by the individual or other user in the carrier in accordance for example with the prescription of a physician treating the individual, and/or in accordance with guidelines of regulatory agency controlling and supervising prescription and over the counter pharmaceutical drugs.

Accordingly, drug delivery devices and related components, compositions methods and systems herein described allow in several embodiments, to provide a patient with a customized combination of drugs specific for the patient within a same device.

Additionally, drug delivery devices and related components, compositions methods and systems herein described allow in several embodiments to perform controlled delivery of one or more drugs possibly in a combination specific for the needs of a patient with increased cost effectiveness, compliance, and patient acceptability compared to other delivery systems, which are expected to increase patient's adherence to efficacy of the therapy. In some of those embodiments the device is in the form of intravaginal ring, diaphragm, pessary, or suppository

Drug delivery devices and related components compositions methods and systems herein described, can be used in connection with various applications wherein controlled release of a drug or combination of drugs at selected effective concentrations is desired. For example, drug delivery devices and related pods, compositions, methods and systems herein described can be used to deliver drugs to an individual to treat or prevent a diseases with a customized combination of effective concentrations or one or more drugs, and in particular to treat or prevent with a single drug delivery system a plurality of diseases in the individual with a combination of effective concentrations of one or more drugs. Additional applications of the drug delivery system herein described comprise drug developments, diagnostics, and fundamental biological research as well as additional applications identifiable by a skilled person upon reading of the present disclosure.

The details of one or more embodiments of the disclosure are set forth in the accompanying drawings and the description below. Other features, objects, and advantages will be apparent from the description and the drawings.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 illustrates an exemplary carrier ring comprising a plurality of openings or receptacles.

FIG. 2 illustrates an opposite side of the ring of FIG. 1, with a smooth surface which comprises no openings.

FIG. 3 illustrates a perspective view of the carrier ring with ten receptacles.

FIGS. 4-5 illustrate an exemplary pod from a top and bottom view.

FIGS. 6-10 illustrate exemplary steps in the fabrication of a pod containing a drug.

FIG. 11 illustrates an exemplary carrier ring with pods.

FIG. 12 shows charts illustrating the results of Tenofovir diphosphate (TFV-DP) administration to vaginal tissues of women with a prior art IVR without removable pods. The charts show vaginal biopsy homogenate TFV-DP levels at 7 days (1205) and vaginal biopsy homogenate FTC levels (1210). Each participant was assigned a Subject ID (indicated in the legend).

FIG. 13 illustrates a cross section of a prior art style pod-ring IVR.

FIG. 14 illustrates a perspective view of a ring assembly.

FIG. 15 illustrates a graph of cervicovaginal lavage concentration (median+SD) of estonogestrel (1510) and ethiny estradiol (1505) delivered simultaneously via combination pod IVRs over 28 days in sheep (n=3). Inverted triangles: estonogestrel; Diamonds: ethiny estradiol.

FIG. 16 panel A shows an exemplary dose-response curve wherein the x-axis shows the ACV concentration in ng/mL and y-axis shows the inhibition percentage. The concentration of acyclovir that inhibits infection by 50% (IC₅₀) when drug was added at the time of HSV challenge and in the overlay medium was ˜700 ng/mL. FIG. 16 panel B shows the median anti-HSV activity of CVL increased from about 31.5% in CVL collected just prior to ring insertion to 57.5% in CVL collected 7 days after ACV IVR. FIG. 16 panel C shows a logarithm reduction in HSV as a function of CVL ACV concentration. The graph suggests the anti-HSV activity of the CVL correlates positively with the drug levels.

FIGS. 17A and 17B illustrate a perspective view of the shell and ring.

FIG. 18 illustrates additional exemplary pods and corresponding carrier rings according to an exemplary embodiment of the drug delivery system herein described.

FIGS. 19A-B show in an exemplary embodiment the dimension of a blank carrier and a corresponding drug pod according to an exemplary embodiment of the drug delivery system herein described.

FIG. 20 illustrates the cumulative release of pritelivir in 50/50 mixture of water and isopropanol provided in vitro in a 28-day period by an exemplary IVR ring with removable pods in accordance with the present disclosure.

FIG. 21 illustrates the cumulative release of octreotide provided in vitro in a 21-day period by an exemplary IVR ring with removable pods in accordance with the present disclosure.

FIG. 22A illustrates in the levels of octreotide in a sheep cervicovaginal lavage in a 28-day preclinical study to test the safety and pharmacokinetics of an exemplary IVR system of the disclosure delivering octreotide in combination with pritelivir. The low-dose IVR consisted of 1 removable pod of octreotide, and 9 removable pods of pritelivir. The mid-dose IVR consisted of 2 removable pods of octreotide, 3 removable pods of pritelivir, and 5 removable pods of carboxymethylcellulose (placebo). The high-dose IVR consisted of 4 removable pods of octreotide, 1 removable pod of pritelivir, and 5 removable pods of carboxymethylcellulose (placebo). The levels of octreotide determined in the sheep CVL samples for the low- (868±757 ng/ml), mid- (2904±1809 ng/ml) and high- (7665±3298) dose IVRs suggests that CVL levels increase with the administered dose.

FIG. 22B illustrates the levels of pritelivir in sheep cervicovaginal lavage in a 28-day preclinical study to test the safety and pharmacokinetics of an exemplary intravaginal ring of the disclosure including formulations delivering pritelivir in combination with octreotide. The low-dose IVR consisted of 4 removable pods of octreotide, 1 removable pod of pritelivir, and 5 removable pods of carboxymethylcellulose (placebo). The mid-dose IVR consisted of 2 removable pods of octreotide, 3 removable pods of pritelivir, and 5 pods of carboxymethylcellulose (placebo). The high-dose IVR consisted of 1 removable pod of octreotide, and 9 removable pods of pritelivir.

### DETAILED DESCRIPTION

The present disclosure describes a drug delivery system compositions methods and systems which allow in several embodiments controlled release of a drug comprised within the pods and/or delivery of a plurality of drugs to an individual to be possibly performed in a combination customizable for the individual based on the for example on the individual medical needs or experimental design.

In some embodiments, the drug delivery system is in the form of an intravaginal ring flexible ring-shaped device (herein also intravaginal ring or “IVR”). The device comprises a plurality of openings into which pods can be inserted. Each pod can contain same or different drugs and allows controlled release, over time, of the drugs of choice. The ring-shaped device of the present disclosure can also be referred to as a torus-shaped device, a ring assembly, a ring device, a carrier ring, or similar terms. The device without pods can be termed as a blank carrier ring.

The term “pod” or “drug pod” as used herein indicates a container configured to hold a drug core (e.g., a coated or uncoated drug tablet) which is typically formed by one or more layers of biocompatible polymers, drug pods can have or not have. In some embodiments the drug pod can further comprise the drug core. Typically drug pods do not have a final sealing polymer layer and can have or not have delivery channel which can be provided by carrier devices configured to hold one or more pods.

In embodiments herein described the blank carrier is typically formed by an impermeable polymer or polymer combination shaped in a configuration that allows formation of receptacles with opening on a surface of the carrier and that will allow positioning of the carrier in a target location of the body of an individual

In some embodiments, a blank carrier ring device herein described, comprising a ring, the ring having a plurality of cylindrical openings, each opening configured to accept a corresponding drug pod, wherein each opening comprises: a recess configured to accept a protrusion in the corresponding drug pod, or a protrusion configured to insert in a recess in the corresponding drug pod. In some embodiments, the plurality of cylindrical opening are located on one or more sides of the ring. In some embodiments, the plurality of cylindrical openings are located on a first side only of the ring.

Corresponding drug pod in accordance with the disclosure comprise a shell, made of an impermeable polymer, having a first base comprising an opening configured to allow delivery of a drug, a second base, a lateral surface, a protrusion attached to, and extending away from, the lateral surface, or a recess in the lateral surface. In some embodiments, the drug pod further comprises a drug core within the shell. In some embodiments the drug pod can optionally further comprise: a semi-permeable or permeable polymer layer on the first base, the semi-permeable or permeable polymer layer covering the opening in the first base,

In general impermeable or semi-permeable polymers materials suitable for fabricating the layers of the blank carrier and/or pods of the drug delivery system include several naturally occurring or synthetic materials that are biologically compatible with body fluids and tissues, such as polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerale opolymer, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer, vinylidene chloride-acrylonitride hydroxypropylmethylcellulose polymer, and ethylcellulose polymer.

Specifically, polymer of the drug delivery system of the present disclosure can be made of any of the above-listed polymers or any other polymer which is biologically compatible with body fluids and tissues, essentially insoluble in body fluids with which the material will come in contact and essentially impermeable or semi-permeable to the passage of the effective agent.

The behavior of any given polymer material as semipermeable, or impermeable (release, blocking, or sealing polymer) can be selected in view of the specific drug or effective agent contained in the core as will be understood by a skilled person. Specifically the permeability of a polymer in a delivery device herein described is dependent on the properties of the drug, such as solubility, hydrophobicity, hydrophilicity or lipophilicity, and log p (octanol-water partitioning coefficient) of the drug in the core of a pod as will be understood by a skilled person.

In particular, the term “impermeable” as used herein in reference to a shell of a pod or blank carrier or other layer of the delivery system refers to a layer that will not allow passage of the effective agent at a rate required to obtain the desired local or systemic physiological or pharmacological effect. Exemplary impermeable polymers comprise silicone, ethylene vinyl acetate copolymer, or polyethylene, or any other polymer which is biologically compatible with body fluids and tissues and essentially impermeable to the passage of the effective agent.

Conversely the term “permeable” as used herein in reference to a shell of a pod or blank carrier or other layer of the delivery system, refers to a layer that will allow passage of the effective agent at a rate required to obtain the desired local or systemic physiological or pharmacological effect.

The term “semi-permeable” as used herein in reference to a layer of a pod herein described refers to a layer that will allow passage of the effective agent, but at a rate significantly slower than if there were no polymer or a release polymer present. “Significantly slower” as used herein refers to a rate 0.5- to 5-log₁₀ units slower. Exemplary semi-permeable polymers comprise for instance, are described in U.S. Pat. No. 4,014,335 which is incorporated herein by reference in its entirety. These materials include cross-linked polyvinyl alcohol, polyolefins or polyvinyl chlorides or cross-linked gelatins; regenerated, insoluble, nonerodible cellulose, acylated cellulose, esterified celluloses, cellulose acetate propionate, cellulose acetate butyrate, cellulose acetate phthalate, cellulose acetate diethyl-aminoacetate; polyurethanes, polycarbonates, and microporous polymers formed by co-precipitation of a polycation and a polyanion modified insoluble collagen. Polylactic acid or cross-linked polyvinyl alcohol is preferred. The semi-permeable coating layer is selected so as to slow release of the agent from the inner core into contact with a mammalian organism, e.g., a human. The semi-permeable coating layer is typically selected to provide gradual release or control of the agent into the biological environment.

In some embodiments, surface reducing or blocking materials (e.g. a blocking polymer or a dialysis membrane) can be introduced in between the semi-permeable layer and impermeable layer to further reduce the surface area and thereby decrease the release rate.

Blocking materials may be placed between the drug core, which may or may not be coated with permeable and/or semi-permeable polymer layer, and the impermeable layer in order to reduce the effective area of the permeable and/or semi-permeable layer available for drug delivery. Examples of such surface-reducing materials are PLA, regenerated, insoluble, non-erodible cellulose, acylated cellulose, esterified celluloses, cellulose acetate propionate, cellulose acetate butyrate, cellulose acetate phthalate, and cellulose acetate diethyl-aminoacetate. In an exemplary embodiment, regenerated cellulose is placed between an uncoated pritelivir pill and a silicone shell containing a 3 mm diameter delivery window. The regenerated cellulose reduces the available surface area, and the reduction of delivery area depends on the pore size of the regenerated cellulose.

In embodiments herein described the pods can optionally comprise one or more layers depending on the desired release of the drug within the drug core and a delivery window to provide passage of the API through the shell and one or more coating forming the drug core.

The term “Optional” or “optionally” as used herein means that a described event or circumstance may or may not occur, and that the description includes instances where said event or circumstance occurs and instances in which it does not. For example, “optionally comprising additional coating” means that an additional coating may or may not be used in the formulation, and that the description includes both the case where an additional coating is present, and the case where it is omitted.

Fore example, drug pod in accordance with the disclosure can comprise a first coating layer formed by a permeable and/or semi-permeable to the API configured to cover at least a portion of drug core, within the shell formed by an additional layer of a polymer which impermeable to the API, configured to cover at least a portion of the first coating layer. In some embodiments, the drug pod herein described can comprise one or more additional coating layer between the first coating layer and the shell coating layer, covering a portion of the first coating layer. Additional layer configuration can be provided by a skilled person in view of the desired drug delivery. In some embodiment, the drug pod can comprise only a shell layer configured to include a drug core.

The term “core” or “drug core” as used herein relating to the drug delivery device refers to a formed (e.g., pressed) API composition (e.g., tablet) that can be coated or uncoated. The term “API” or active pharmaceutical ingredient, as well as the term bulk active and active substance as used herein indicate the ingredient in a pharmaceutical drug or natural product that is biologically active. In a drug core one or more API are typically comprised together with one or more suitable excipients. “Excipient” as used herein refers to pharmaceutically inactive components included in the drug delivery device. Excipients comprise to dyes, flavors, binders, emollients, fillers, lubricants, and preservatives. Selection of excipients in a drug core can be used to modify the release or other characteristics of the API; for example, increasing the solubility of the API, or they may be inactive, such as fillers and colorings as would be identifiable by a skilled person.

The wording “may” as used in the present disclosure is used interchangeably with the word “can” to indicate operability of a referenced item, the ability of a referenced item to perform one or more functions and/or activities, and/or inclusion of a referenced item within the scope of the disclosure, according to the related context as will be understood by a skilled person upon reading of the disclosure.

In drug delivery system herein described one or more drug cores can contain more than one active ingredient. The traditional word for the API is pharmacon or pharmakon (from Greek: φ\acute{α}ρμακoν, adapted from pharmacos) which originally denoted a magical substance or drug. Exemplary API or classes thereof that can be delivered in a core comprise atazanavir, didanosine, efavirenz, emtricitabine, lamivudine, lopinavir, nevirapine, raltegravir, ritonavir, saquinavir, stavudine, tenofovir, tenofovir disoproxil fumarate, zidovudine, acyclovir, famciclovir, valcyclovir, morphine, buprenorphine, estrogen, progestin, progesterone, cyclosporine, a calcineurin inhibitor, prostaglandin, a beta-blocker, gentamycin, corticosteroid, a fluoroquinolone, insulin, an antineoplastic drug, anti-nausea drug, a corticosteroid, an antibiotic, morphine buprenorphine, a VEGF inhibitor Leuprolide, Octreotide, Buprenorphine, Naloxone, Tenofovir disoproxil fumarate, Emtricitabine, Acyclovir, Pritelivir, Dapivirine, Dolutegravir, Rilpivirine, Cabotegravir, Liraglutide, Maraviroc, Levonorgestrel, Etonogestrel, Ethinyl estradiol, Teriparatide, Tacrolimus.

Cores in the sense of the disclosure can be uncoated or coated with a permeable polymer.

Additional examples of classes of agents can be incorporated into a drug core in accordance with the present disclosure as the API comprise anesthetics and painkilling agents; antiallergenics, antibiotics, antibacterials, anticancer or antineoplastic agents such as chemotherapeutic agents and antiproliferative agents, anti-inflammatory agents, anti-fungal agents antiviral cell transport/mobility impending agents Beta-blockers, Prostaglandins, decongestants, HIV drugs hormones immunological response modifiers, miotics and anti-cholinesterase mydriatics peptides and proteins steroidal compounds, sympathomimetics, carbonic anhydrize inhibitors, agents to treat incontinence; and agents commonly used in topical therapy, agents that act on the sympatheic and/or parasympathetic nervous systems; antipsychotics; and antidepressants, and additional API indicated in can be found in U.S. application Ser. No. 14/124,517 issued as U.S. Pat. No. 9,937,335 which is incorporated herein by reference in its entirety.

In embodiments herein described the core within pods described herein are held in place by the carrier ring in a configuration presenting the opening of the pods to the body parts of the individual which are the target of the drug delivery. In some embodiments, pods comprise solid pills of compressed drug or active pharmaceutical ingredient (API)-coated with a permeable polymer membrane or uncoated to form a core. In some embodiments, the cores can comprise the API without being coated with a permeable or semi-permeable polymer. These cores can be placed in silicone shells with a release window facing the vaginal fluids. Water crosses the permeable membrane forming a saturated solution of drug within the core. The concentration gradient formed across the pod membrane drives diffusion of the drug through the release window and into the vaginal fluid, resulting in pseudo-zero order (linear) release. This design allows release from the IVRs to be controlled for drugs spanning a wide range of aqueous solubilities, spanning the range from hydrophobic and hydrophilic small molecules to high molecular weight, highly-soluble biomolecules. This is a particularly advantageous feature given the proposed IVR combination includes drugs of varying solubility (for example, drugs such as acyclovir (ACV) are extremely water soluble, whereas drugs such as Etonogestrel (ETG) and Ethinyl Estradiol (EE) are highly hydrophobic).

In some embodiments, human-sized carrier-IVRs can accommodate up to 10 pods but more or less pods can be accommodated in view of the ring configuration and/or related intended use. However, any number of pods can be employed. The carrier ring can comprise a higher or lower number of pods, and each pod may be used in any one ring, or some slots in the ring may be filled with a blank pod which does not contain any drug. In a drug delivery system, each of the pod can contain cores with up to 70 mg of drug substance, typically about 60-70 mg of drug in formulations further comprising one or more excipients. By tailoring the drug concentration in the drug pods and the pod-IVR release parameters, the delivery rate can be precisely controlled over as many as three orders of magnitude. For example, different formulations of Tenofovir disoproxil fumarate (TDF), in a TDF pod-IVRs can be delivered by altering the polymers and delivery areas. For example, the release rate can vary between 10 μg/d to 13 mg/d. The carrier ring, being merely a support structure, does not have an impact on drug release rate. The flexibility of the pod-IVR approach can also be aimed at delivering combinations of new, experimental Antiretroviral (ARV) agents including biomolecules. Pod-IVRs delivering antiviral drugs have been evaluated in the mouse, rabbit, macaque, and sheep models. A pod-IVR demonstrating PK of a pilot multipurpose pod-IVR simultaneously delivering five drugs (three ARVs and two hormonal contraceptives—ETG and EE) in an ovine model showed proof-of-concept of the IVR platform described herein. Pilot rings releasing Tenofovir disoproxil fumarate (TDF) and Emtricitabine (FTC) (combination herein also indicated by TDF-FTC) and acyclovir in women have been tested.

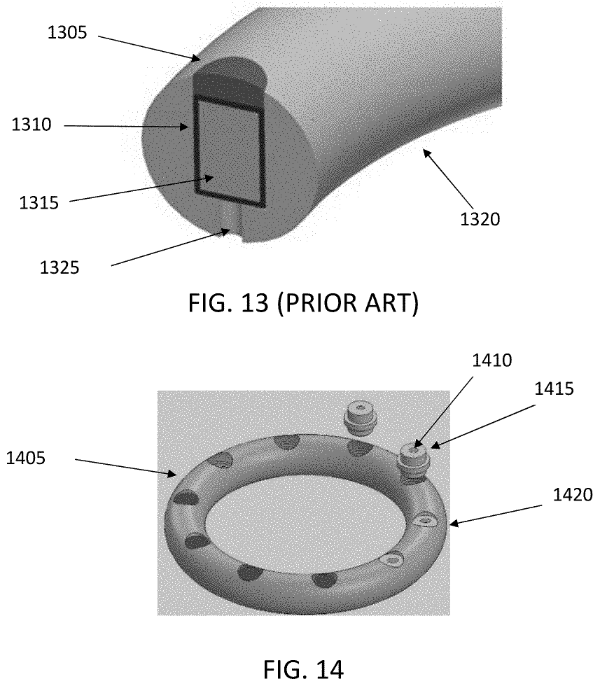

In prior “pod-in-ring” technology the drug delivery release window was formed as part of the ring, as visible in FIG. 13. FIG. 13 illustrates a cross section of a prior art style pod-ring IVR. The drug delivery window (1325) is fashioned in the ring (1320). The pod (1310, 1315) is placed in a receptacle with a delivery window pre-formed into the ring body. The receptacle is then back-filled with silicone (1305). The disadvantage of this design is that the “regulatory unit” is the ring. Approval of a TDF ring for HIV pre exposure prophylaxis (HIV-PrEP) and an ETG-EE ring for contraception would not allow prescription for both indications without the need of new, large, and very costly trials.

The technology of FIG. 13 can be modified into a replaceable “shell-based” pod-IVR platform where the drug delivery window (1410) is incorporated into each pod (1415). The pods can be inserted in receptacles (1420) or openings in the carrier ring (1405). The different structure of the delivery system in FIG. 14 relative to the device of FIG. 13 presents technical changes which lead to great regulatory advantages. With a drug delivery systems herein described now possible to first acquire approval of a blank “carrier” ring (1405) using a 510(k) or premarket approval (PMA) device approval route. The blank carrier ring, while having no drug within, will have receptacles to allow incorporation of one or more self-contained drug delivery pods. In some embodiments, API-containing drug pods have a delivery channel at the top (1410)—the surface area of this channel is one of the critical factors affecting release rate, and can be chosen and controlled during fabrication. For patients that require less drug pods than a carrier contains, empty ring cavities can be filled with blank or non-drug containing pods (e.g. carboxymethylcellulose pods) in order to maintain the surface smoothness of the carrier ring.

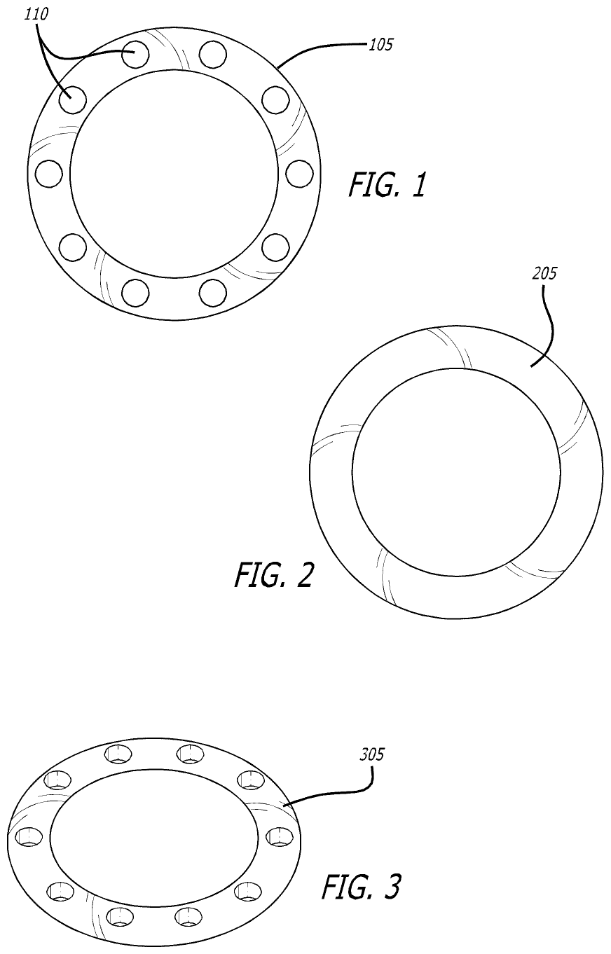

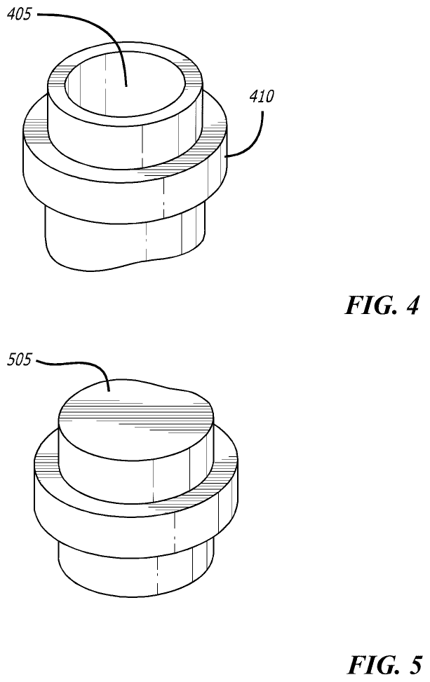

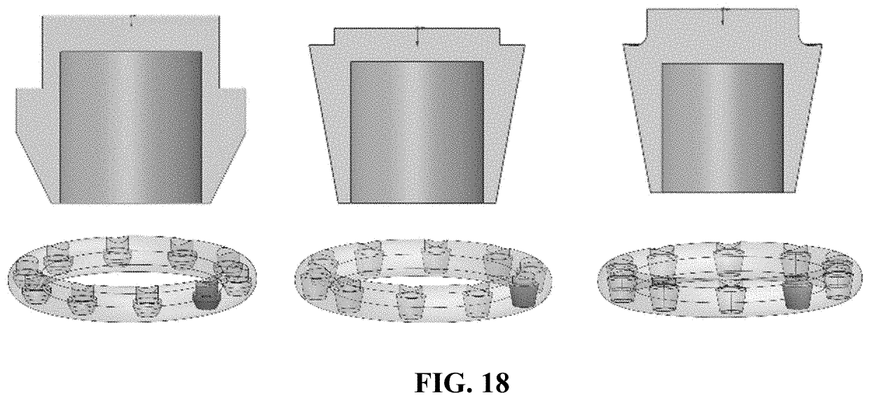

FIG. 1 illustrates an exemplary carrier ring (105) comprising a plurality of openings or receptacles (110) in accordance with the present disclosure. In some embodiments, ten receptacles are present in the carrier ring. FIG. 2 illustrates an opposite side to that of FIG. 1, with a smooth surface which comprises no openings. FIG. 3 illustrates a perspective view of the carrier ring with ten receptacles. The number of receptacles is exemplary, and in other embodiments more or less receptacles of a same or different configuration can be used. In some embodiments the receptacles and corresponding pods can have a same configuration (e.g. the configuration of FIG. 4). In some embodiments the receptacles and corresponding drug pods can have different configurations (e.g. a combination of configurations shown in FIG. 4 and FIG. 18) for example to identify pods comprising different drugs and/or different drug concentrations. In some embodiments, the carrier ring, the shells or both are transparent in the optical wavelength range.

FIGS. 4-5 illustrate an exemplary pod shell from a top and bottom view. In FIG. 4 an opening (405) of the pod shell allows insertion of a drug core into the shell. In FIG. 5, the opposite side of the receptacle of FIG. 4 is shown, comprising a closed surface (505) which allows retention of the drug core within the shell.

In some embodiments, the opening (405) has a diameter about 4 mm and height of about 5 mm to allow an accommodation of about 60-70 mg of the drug substance and a maximum release rate per pod as high as 2 mg/day, or 10 mg/day or higher depending on the pod configuration as well as configuration of the related drug core.

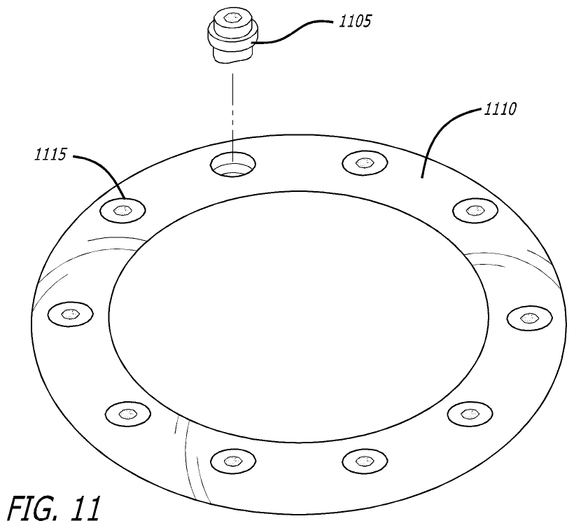

The side protrusion (410) illustrated in FIG. 4 is designed in a way such that the pod does not pop out of the ring when bended. Instead, the pod should be retained within the ring under controlled environments such as in in vitro dissolution tests or in vivo animal studies. The side protrusion can be cylindrically shaped or otherwise shaped so as to be self-anchoring to the carrier. Exemplary blank carriers and corresponding drug pods are illustrated in FIG. 11 and FIG. 18. Typically, the side protrusion (410) is about 0.5-1 mm thick.

Different configuration of the pods can comprise protrusions and corresponding recess having different shapes and dimensions and which are configured to allow retainment of the pod within a carrier ring of choice.

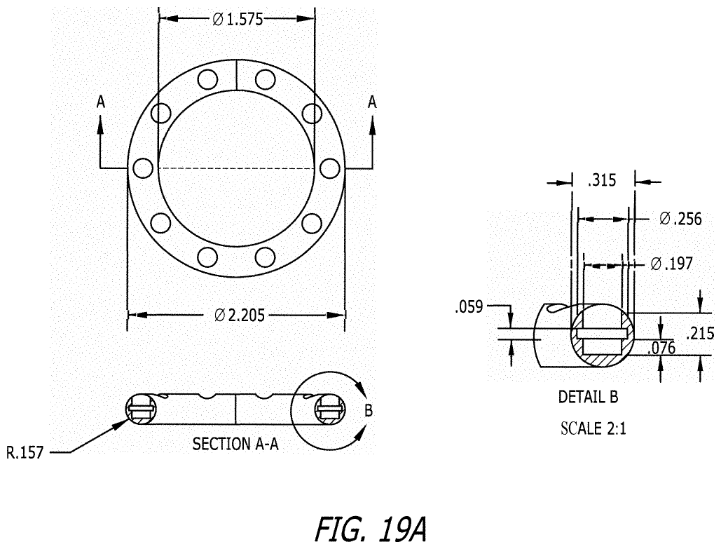

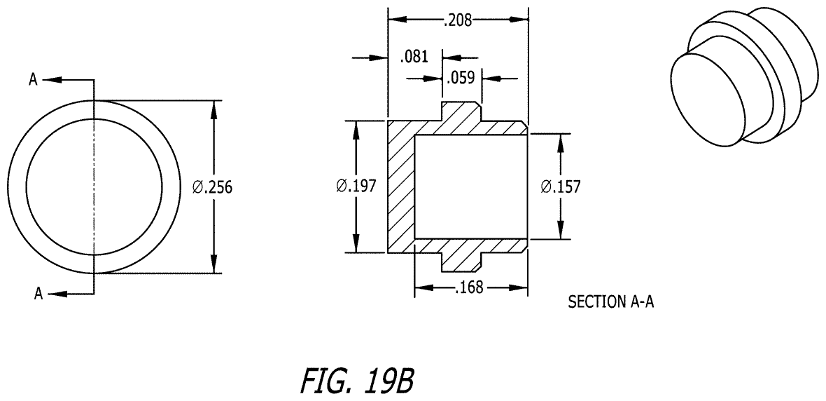

FIGS. 19A-B shows in an exemplary embodiment the dimension of a blank carrier and a corresponding drug pod. In particular, FIGS. 19A-B show a blank carrier ring with outer and inner diameters of 2.205 and 1.575 inches, respectively. The pod shell has an inner diameter and height of 0.157 and 0.168 inches, respectively.

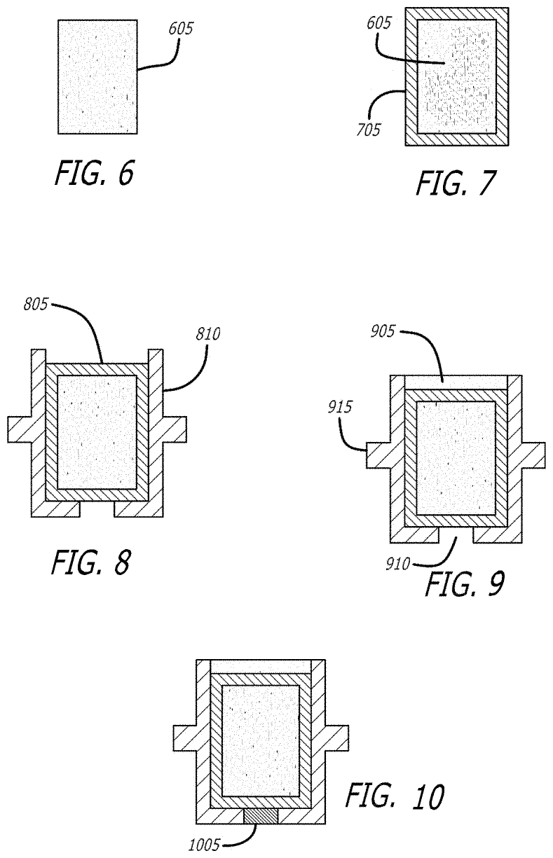

FIGS. 6-10 illustrate exemplary steps in the fabrication of a pod containing a drug. A drug pod comprises an outer shell and a drug core inside the shell. The drug core comprises a pill coated with polymer. In other embodiments, the pill may be without a polymer covering. For example, FIG. 6 illustrates a pill (605). The pill can be manufactured by mixing the API with excipients and by pressing the ingredients into the shape of the inner volume of the pod shell. In this example, the inner volume of the pod shell is a cylinder, therefore the pill is formed in a cylindrical shape.

In a second step, the pill is coated with a polymer (705) which is semi-permeable. For example, poly(vinyl alcohol) (PVA) may be used as a polymer coating. The polymer, being semi-permeable, allows release of the active ingredients in the pill (605). The pill coated with the polymer forms a drug core. In some embodiments, the drug core comprises only the API, without the semi-permeable polymer.

As illustrated in FIG. 8, the drug core (805) is inserted in a shell (810) during shell assembly. The shell, in some embodiments, is made of silicone or another impermeable polymer. The carrier ring and shells can be made of the same material. The API can therefore be released through the semi-permeable polymer coating but cannot pass through the impermeable polymer.

FIG. 9 illustrates a successive step in the drug pod assembly, back-filling the receptacle with a material such as a silicone adhesive (905). The adhesive is impermeable to the drug and allows safe attachment of the drug core to the shell, as well as preventing drug diffusion. As visible in FIG. 9, the shells, in some embodiments, comprise side protrusions (915), as well as an opening (910). The side protrusions are also illustrated in FIG. 4 (410). In this example, the side protrusions are cylindrically shaped and allow safe retention of the shells within the ring, as the flexible protrusion are inserted in the corresponding groove in the ring, and retain sufficient rigidity to resist accidental removal.

In a successive step illustrated in FIG. 10, the opening in the shell is filled with a semi-permeable material (1005), such as a hydrogel. For example, the same polymer of layer (705) in FIG. 7 can be used. Referring back to FIG. 10, the material (1005) acts as a wicking layer as it acts as a delivery window for the drug contained in the pod. The size of the opening, and therefore the size of the wicking layer, can be adjusted depending on the desired drug release rate. In this example, the openings are circular in shape.

FIG. 11 illustrates an exemplary carrier ring with pods. A carrier ring (1110) is illustrated, with filled pods (1115) as well as a pod outside the ring (1105) for illustrative purposes. In some embodiments, the shell of each pod can be made using standard injection molding techniques. The pill can be fabricated with a mixture of an active pharmaceutical ingredient (API) and an excipient (such as magnesium stearate, carboxymethylcellulose, etc.). The mixture can be tableted using a standard tablet press—a method commonly used in the manufacturing industry. The resulting pill is cylindrical in shape—the diameter of the pill depends on the diameter of the die of the tablet press; the height of the pill depends on the amount of API-mixture being pressed. The pill can then be coated with a semi-permeable polymer (PVA) either by, for example, dip-coating, spray coating or pipetting some polymer over the pill and allowing it to dry for few hours.

In some embodiments, the drug core can be manually inserted in the shell, and the open end back-filled with silicone adhesive. However, automated fabrication can also be employed. A delivery channel (or delivery window) can be pre-punched (i.e. prior to insertion of the drug core) on the side opposite to the back-filled end of the shell. In some embodiments, the delivery channel can be created with a biopsy punch after the shell has been back-filled. The drug pods can be easily inserted on a blank carrier ring, for example by the dispensing pharmacist or the patient.

In any of the embodiments described in the present disclosure, the API can be covered by a permeable polymer, either fully or partially covered. In some embodiments, the permeable polymer may cover, for example, only one surface of an API pill. For example, the pill may be cylindrical in shape, and may be covered by a permeable layer only on a top, circular surface, corresponding to the release window of a pod. The remaining surfaces of the API pill, in some embodiments, may be covered by the impermeable polymer of the drug pod, thereby preventing any significant drug release other than that occurring through the release window of a pod.

In any of the embodiments described in the present disclosure, the drug release devices may be modified so as to obviate any need for covering the API in a permeable polymer. For example, the drug pill may be prepared with the API without being covered by a permeable polymer. In a separate step, a permeable or semipermeable polymer layer may be deposited on an inner surface of the removable pod. The API-only pill can then be inserted, manually or automatically, into the pod. This embodiment presents the advantage of separating the permeable polymer application from the preparation of the pill. For example, the permeable polymer to control the release rate of the drug may require curing, for example at a specific temperature. This temperature may adversely affect the drug. If the permeable polymer is deposited on the pill and then cured, the API may lose some efficacy due to the thermal treatment. However, if the permeable polymer is deposited on the pod, specifically blocking the release window on an inner surface, the polymer can be thermally cured before insertion of the pill. In this way, the API remains unaffected by the thermal treatment required for curing certain polymers used for controlling the drug release rate. In an optional step, a wicking layer can also be added in the delivery window of the pod. In some embodiments, the permeable polymer may also be partially cured within the release window.

The API-excipient ratio, desired pill height, polymer concentration, polymer coating process, diameter of the delivery window, and the number of drug pods to be inserted in the carrier ring can be varied depending on the target drug release rate. In some embodiments, poly(lactic acid) (PLA) or other similar surface reducing or blocking materials (e.g. a blocking polymer or a dialysis membrane) can be introduced in between the semi-permeable layer and silicone to further reduce the surface area and thereby decrease the release rate. In these embodiments, the impermeable material is coated onto the permeable layer (705) of FIG. 7, leaving an opening corresponding to the opening (910) in the shell. The opening of this additional impermeable material will be smaller, and effectively allows a smaller delivery window to remain in the drug core instead of modifying the opening size of the shell, or additionally to the modification of the opening size of the shell. The additional impermeable opening may be deposited only on the surface of the drug core corresponding to the opening in the shell, or entirely around the drug core—in both cases leaving an opening to allow for drug delivery.

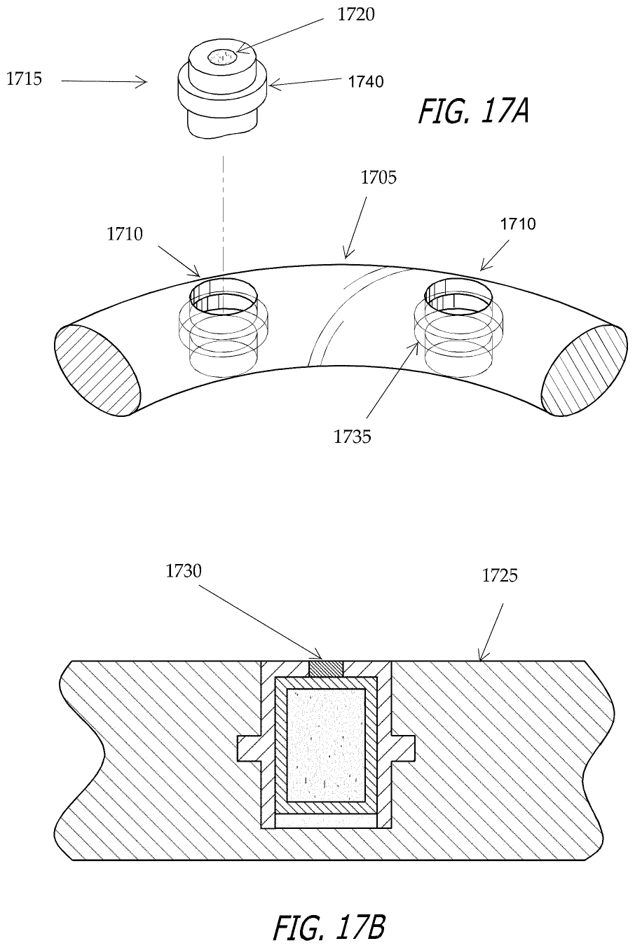

FIG. 17A illustrates a section of a ring (1705) with two receptacles (1710), and a shell (1715) to be inserted in the ring. It can be noted that an opening in the shell, closed off with a wicking material, is visible (1720). The wicking material disk allows the drug to be released at a controlled rate. The ring comprises an annular recess (1735) to accept the annular protrusion (1740) of the shell. FIG. 17B illustrates a cross sectional view of part of a ring (1725), with a shell within the corresponding receptacle. The wicking disk (1730) is also illustrated. The layers of the shell in FIG. 17B are described in FIGS. 6-10.

In some embodiments, the drug pills are coated to produce cores with PVA or PLA polymer using published dip-coating methods. A spray coater may also be used. In some embodiments, prior to pod assembly, silicone shells blanks are cleaned and all subsequent assembly steps are performed in a laminar flow (downdraft) hood. The empty shells can have pre-punched delivery channels. The channel size is dependent on the coated drug core placed in that cavity. After placement of the coated pills in the blank shells, it can be back-filled using a silicone adhesive. The backfill adhesive can, for example, be cured 24 h in an oven at 40° C., and the pods can be packaged in sterile pouches prior to removal from the laminar flow hood. As in all commercial IVRs the products are clean and without endotoxins or residuals, but need not be sterile.

In some of these embodiments, the API is in a powder form, and is compressed, for example in a tablet press, into a pill form. For example, the API can be compressed into a cylindrical pill. In some embodiments, the API may comprise Leuprolide (for endometriosis, uterine fibroids, infertility, or contraception), Exenatide (for diabetes), Octreotide (for acromegaly, or other neuroendocrine tumors), Buprenorphine (for pain addiction), Naloxone (for pain addiction), Tenofovir disoproxil fumarate (for TDF) (for HIV PrEP), Emtricitabine (for FTC) (for HIV PrEP), Acyclovir (for herpes), Pritelivir (for herpes), Dapivirine (for HIV PrEP), Dolutegravir (for HIV PrEP/treatment), Rilpivirine (for HIV PrEP/treatment), Cabotegravir (for HIV PrEP/treatment), Liraglutide (for diabetes), Maraviroc (for HIV PrEP), Levonorgestrel (for contraception), Etonogestrel (for contraception), Ethinyl estradiol (for contraception), Teriparatide (for osteoporosis), Tacrolimus (for obliterative vulvovaginitis). Other APIs may be used, for example as described in the present disclosure.

In some embodiments, the pill is then covered by a permeable or semi-permeable polymer. In some embodiments, the pill is not covered by the polymer, which is instead placed in the removable pod. In some embodiments, both the pill and the pod may comprise a permeable polymer to control the release rate. The person of ordinary skill in the art will understand that whether a polymer is permeable or semi-permeable may vary according to the specific API under consideration. The present disclosure may refer to a polymer to control the release rate of the API as either a permeable or semi-permeable polymer. For the embodiments which coat the API with a polymer, different methods may be employed, such as, for example, dip-coating, drop-coating, or fluid-bed coating.

In some embodiments, a semi-permeable polymer used to coat the API can comprise PVA (2 to 10%), polyurethane, or polycaprolactone. In some embodiments, the purpose for coating the pill with a semi-permeable polymer is to hold the pill together. In other cases, the polymer performs the added function of modulating drug release. In some embodiments, the polymer may perform both functions of preserving the mechanical integrity of the pill as well as controlling the release rate of the drug.

In some embodiments, the material used for the wicking layer, also referred to as the “wicking material” is selected to modify the rate of transport of API through the delivery window. Depending on the wicking material, the drug and the application, the rate of transport can be increased or decreased. For example, heat treated PVA decreases release rate for acyclovir and PEG increases the rate for pritelivir. The wicking material can comprise a hydrophilic polymer or a fiber material. In various embodiments, the fiber material can be selected from the group consisting of silk, cotton, Nafion and combinations thereof. In various embodiments, the wicking material can comprise carboxymethylcellulose-hydroxyethylcellulose (“CMCHEC”) copolymer. In various embodiments, the wicking material can comprise polyvinylalcohol-acrylate (“PVA-MA”) copolymer. In various embodiments, the wicking material can comprise polyethylene glycol-methacrylate copolymer.

In some embodiments, the wick can completely fill the delivery window space, or it may only partially fill the window. Partial filling can be in the center of the window passage (such as a fiber penetrating the window), or it can be a wicking polymer coating on the delivery window inside walls. In some embodiments, the hydrogel material, besides filling the delivery window, may also partially fill the drug pod cavity, providing a hydrogel layer completely surrounding, or partially surrounding, the drug core.

The wicking material can also be chemically bound to the impermeable layer of the pod as opposed to simply coating the surface. An example of this would be modification of the exposed silanol (Si—OH) functionalities on the delivery window walls using a poly(ethylene glycol) cross-linked polymer to improve surface wetting.

Detailed description about the characteristics and additional configuration of the wicking material applied within the drug cavity and/or delivery window can be found in U.S. application Ser. No. 14/124,517 issued as U.S. Pat. No. 9,937,335 which is incorporated herein by reference in its entirety.

In some embodiments, the shell will have a cylindrical shape, with a first base comprising an opening for drug delivery, an empty second base, and a lateral surface. The second base may be sealed off during manufacturing, with an adhesive such as a silicone adhesive, thus allowing insertion of the drug pill. In some embodiments, the size of the opening, and therefore the drug release rate, can be controlled during fabrication by punching the shell surface with a punching tool of different diameter. In this way, different shells can be advantageously produced without radical changes to the fabrication process. In other embodiments, the shell may have different shapes, such as non cylindrical shapes. In some embodiments, the protrusion in the shell of the drug pod, and the corresponding recess in the receptacles or openings of the ring, may have different shapes, such as, for example, annular, cylindrical, or others.

In some embodiments the protrusions and corresponding recesses may be reversed, with the protrusion being within the ring device, and the recesses in corresponding positions in the shells to be inserted in the ring device. Different types of protrusions and recesses may be used. The protrusion and recess system described above for different embodiments may be referred to as a snap fit. For example, the ring device may be described as having snap fit means, and the shells may have corresponding snap fit means. The rigidity of the materials used may be adjusted accordingly so that in some embodiments the insertion of the shells in the ring may be “soft” while in other embodiments it may include a “snap”. In some embodiments, the ring device may have a modified shape. For example, the ring device may comprise a membrane in the center.

The behavior of any given polymer material as semipermeable, or impermeable (release, blocking, or sealing polymer) is dependent on the properties of the drug, such as solubility, hydrophobicity, hydrophilicity or lipophilicity, and log p (octanol-water partitioning coefficient) of the drug in the core of a pod as will be understood by a skilled person.

Drug release from the delivery devices disclosed herein is be controlled by multiple factors, including the solubility of the drug in the release fluid, the polymer coatings applied to the drug core, the size and quantity of delivery window channels exposing the drug core to the release fluid, and the characteristics of any wicking materials applied within the drug cavity and/or delivery channels.

A large number of polymers which are inert, non-immunogenic and of the desired permeability can be used to construct the devices described in the current disclosure. which are inert, non-immunogenic and of the desired permeability.

For example, an impermeable layer of the device of the present disclosure can be made of an impermeable members of above-listed polymers, preferably silicone, ethylene vinylacetate copolymer, or polyethylene, or any other polymer which is biologically compatible with body fluids and tissues and essentially impermeable to the passage of the effective agent.

A permeable and/or semi-permeable layer as well as an optional wick material of the pod and/or delivery system herein described is can be made of an appropriate permeable and/or semi-permeable polymer for example apolyvinyl alcohol hydroxyethylcellulose carboxymethylcellulose copolymer, polyvinyl alcohol-methacrylate copolymer, or polyethylene glycol-methacrylate copolymer which is biologically compatible with body fluids and tissues and permeable to the passage of the agent or composition effective in obtaining the desired effect.

The configuration of the pod and drug delivery system herein described can be performed to control the release of an API into an individual to desired target locations (e.g. organs or tissues) at desired effective target concentrations. For example, the configuration of the blank carrier and related number and position on the rings of the receptacles can also be performed in light of the number of pods to be hosted and target locations (e.g. organs and tissues) where the drug is intended to be released. Also the number of layers and corresponding selected combination of impermeable, permeable and/or semi-permeable polymers, the presence or absence of a wick and selection of corresponding permeable polymer, as well as configuration of the delivery window structure and possible crosslinking of selected polymer can be performed in function of a desired concentration of the drug to be released by the delivery system and related intended systemic or topical route of administration.

The wording “systemic administration” as used herein indicates a route of administration by which the active principle is brought in contact with the body of the individual so that the desired effect is not necessarily limited to the specific tissue where the drug is released. The wording “topical administration” as used herein relates to a route of administration wherein the active agent usually included in an appropriate formulation directly where its action is desired.

In particular, a person skilled in the art will understand that the drug-polymer combination can be selected and modulated to control drug dissolution and subsequent release rate in order to reach a desired target concentration on a target delivery site in the body which results in the intended systemic and/or topical administration route for the one or more drugs delivered.

The modulation of release rate can be achieved by a combination of factors as will be understood by the skilled person. For example, the release rate can be increased by adding excipients, such as carboxymethylcellulose, or PEG, reducing the semi-polymer coating thickness, making the semi-polymer coating layer less cross-linked, increasing the delivery window area, and/or increasing the total number of removable pods in case that the pod has a threshold maximal release rate. The release rate can also be decreased by increasing the semi-polymer coating thickness, cross-linking the semi-polymer coating layer and reducing the delivery window size.

Suitable polymer materials can be selected for drugs with various thermal stability rates. For example, semi-permeable polymers such as PVA are preferable for drugs exhibiting higher thermal stability even beyond 190° C. such as acyclovir. For drugs that are not heat-stable beyond 190° C. such as leuprolide, semi-permeable polymers such as PLA or regenerated cellulose can be used to reduce its release rate. For drugs with low solubility such as pritelivir, a permeable polymer or no polymer is preferred to improve the pill's structural integrity.

In some embodiments, the drug device herein described is adapted for intravaginal administration. The desired release rate for each removable pod depends on the vaginal bioavailability of the drug. The pod is configured and adjusted to deliver the API at a therapeutic level. For example, an implant which subcutaneously delivers a drug at 50 μg/day achieves plasma levels of 50 ng/ml which is considered to be therapeutic. However, when the same drug is intravaginally delivered at the same release rate through an intravaginal ring containing drug pods, only a plasma levels of 5 ng/ml is attained, i.e. approximately 10% vaginal bioavailability. Therefore, the intravaginal ring and the drug pods will need to be reconfigured to release 10 times faster to result in similar plasma levels as the implant.

Examples of how to prepare intravaginal rings with selected polymer materials and combinations to deliver specific drugs at a desired target release rate can also be found in the U.S. Ser. No. 14/124,517. In particular, Example 1 of the U.S. Ser. No. 14/124,517 application describes experimental protocols for designing an intravaginal ring containing a cellulose-based polymer hydrogel Carboxymethylcellulose-hydroxyethylcellulose copolymer (CMC-HEC) that releases the microbicide drug tenofovir (TFV). In this example, the hydrogel acts as a wick filling the delivery window, drawing water into the drug pod and removing the lag time for release. Example 2 of the U.S. Ser. No. 14/124,517 application describes details about an intravaginal ring with polyvinylalcohol-acrylate (PVA-MA) co-polymer hydrogel for delivering tenofovir. The PVA-MA hydrogels do not release TFV as rapidly as do otherwise identical IVRs with the CMC-HEC hydrogel. The PVA-MA hydrogels, however, can be cross-linked in situ in the delivery window to allow the extent of cross-linking to be controlled, and allow much more precise control of drug release rate by modifying the hydrogel.

In delivery systems herein described, the effective agent diffuses in the direction of lower chemical potential, i.e., toward the exterior surface of the device. At the exterior surface of the device, equilibrium is again established. When the conditions on both sides of the permeable and/or semi-permeable coating layer are maintained constant, a steady state flux of the effective agent will be established in accordance with Fick's Law of Diffusion. The rate of passage of the drug through material by diffusion is generally dependent on the crystallinity of the polymer (the higher the cross linking, the higher the crystallinity and the lower the permeability) and on the solubility of the drug therein, as well as on the thickness of the wall, among other parameters (see below). This means the selection of appropriate materials for fabricating the wall will be dependent on the particular drug to be used.

The rate of diffusion of the effective agent through a polymeric layer of the present disclosure may be determined via diffusion cell studies carried out under sink conditions. In diffusion cell studies carried out under sink conditions, the concentration of drug in the receptor compartment is essentially zero when compared to the high concentration in the donor compartment. Under these conditions, the rate of drug release is given by:

$$\frac{Q}{t} = \frac{D \times K \times A \times DC}{h}$$

where Q is the amount of drug released, t is time, D is the diffusion coefficient, K is the partition coefficient, A is the surface area, DC is the difference in concentration of the drug across the membrane, and h is the thickness of the membrane.

In the case where the agent diffuses through the layer via water filled pores, there is no partitioning phenomena. Thus, K can be eliminated from the equation. Under sink conditions, if release from the donor side is very slow, the value DC is essentially constant and equal to the concentration of the donor compartment. Release rate therefore becomes dependent on the surface area (A), thickness (h) and diffusivity (D) of the membrane. In the construction of the device of the present disclosure, the size (and therefore, surface area) is mainly dependent on the size of the effective agent.

Thus, permeability values may be obtained from the slopes of a Q versus time plot.

The permeability P, can be related to the diffusion coefficient D, by:

$$P = \frac{K \times D}{h}$$

In some embodiments, of drug delivery systems herein described a drug pod comprises: a shell, made of an impermeable polymer, having a first base comprising an opening configured to allow delivery of a drug, a second base, a lateral surface, and further comprising: a crosslinked semi-permeable polymer layer on the first base, the semi-permeable polymer layer covering the opening in the first base. In some embodiments, the drug pod further comprises a drug core within the shell.

Several embodiments of the disclosure, a drug delivery system can comprise: a blank carrier ring device and at least one drug pod configured to be inserted in a receptacle of the plurality of receptacles, the at least one drug pod comprising: a shell, made of an impermeable polymer, having a first base comprising an opening configured to allow delivery of a drug, a second base, a lateral surface, and a cross-linked semi-permeable polymer layer on the first base, the semi-permeable polymer layer covering the opening in the first base, and a drug core within the shell.

Several embodiments of the disclosure also provide for a method of treating an individual to obtain a desired local or systematic physiological or pharmacological effect from the API.

The term “individual” as used herein includes a single animals having system and/or apparatuses configured to host a delivery system herein described, for example higher animals and in particular vertebrates such as mammals and in particular human beings.

The method comprises providing a drug delivery system described herein wherein the drug delivery system comprises plurality of drug cores within a plurality of drug pods, each drug core of the plurality of drug core within a corresponding pod of the plurality of drug pods, each drug core of the plurality of drug core and corresponding pod selected to provide the individual with one or more target active pharmaceutical ingredient at one or more effective target concentrations.

In particular, each drug pod can be configured by selecting a combination of polymers and related layer configured to release the one or more target drug with one or more target release rate.

The method further comprises administering the drug delivery system to the individual to deliver one or more target drug to the individual. In particular the administering the drug delivery system to the individual and allowing the API to pass through the delivery window to contact the individual. Administering can include positioning, inserting, injecting, implanting, or any other methods for exposing the drug delivery device to the individual.

The drug delivery device of the present disclosure can also be administered to a mammalian organism via any topical or systemic route of administration known in the art. Such routes of administration include intraocular, oral, subcutaneous, intramuscular, intraperitoneal, intranasal, dermal, and the like. In addition, one or more of the devices may be administered at one time or more than one agent may be included in the core.

In various embodiments, a disease condition is treated or prevented through the devices of the present disclosure. In various embodiments, the disease condition can be selected from the group consisting of vaginal condition, uterine condition, pelvic condition, rectal condition, eye condition, ear condition, sinus condition, nasal condition, prostatic condition, and bladder condition.

The term “treatment” as used herein indicates any activity that is part of a medical care for, or deals with, a condition, medically or surgically.

The term “prevention” as used herein indicates any activity which reduces the burden of mortality or morbidity from a condition in an individual. This takes place at primary, secondary and tertiary prevention levels, wherein: a) primary prevention reduces the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “disease”, “condition” or “disease condition” as used herein indicates a physical status of the body of an individual (as a whole or of one or more of its parts), that does not conform to a standard physical status associated with a state of complete physical, mental and social well-being for the individual. Conditions herein described comprise a condition of the living individual that is associated to a functional abnormality of the body or of any of its parts, and a condition of the living individual that impairs normal functioning of the body or of any of its parts and is typically manifested by distinguishing signs and symptoms.

In various embodiments, one or more disease conditions can be treated or prevented with a drug delivery system herein described and can selected from the group consisting of hyperhomocysteinemia, chronic renal failure, end stage renal disease, hemodialysis, peritoneal dialysis, vascular dementia, cardiovascular disease, stroke, cerebrovascular accidents, thrombotic disorder, hypercoagulable states, venous thrombosis, deep vein thrombosis, thrombophlebitis, thromboembolic disease, ischemic stroke, restenosis after percutaneous transluminal coronary angioplasty (PTCA), preeclampsia, vasculitis, digital ischemia, multifocal osteonecrosis, retinal vein occlusion, glaucoma, miscarriage, pregnancy complication, placental abruption, transplantation, diabetic retinopathy, ischemic bowel disease, cerebral vein thrombosis, atherosclerosis, coronary artery disease, penile venous thrombosis, impotence, central venous thrombosis, peripheral artery disease, intermittent claudication, hemorrhagic colitis, radiation enteritis, radiation colitis, visceral ischemia, acute mesenteric ischemia, chronic mesenteric ischemia, hypertension, microangiopathy, macroangiopathy, recurrent leg ulcer, carotid stenosis, occlusive vascular disease, arterial aneurysm, abdominal aortic aneurysm, congestive heart failure, hepatopulmonary syndrome, high flow state associated with chronic liver disease, migraine headache, vascular headache, dizziness, lightheadedness, orthostatic intolerance, postural hypotension, postural hypotension, postural orthostatic tachycardia syndrome, idiopathic pulmonary fibrosis, pulmonary hypertension, angioedema, vaso-vagal faints, neuroleptic malignant syndrome, learning disorder, learning disability, insomnia, dementia, age associated memory impairment, attention deficit/hyperactivity disorder (ADHD), mild cognitive impairment, Alzheimer's disease, Down's syndrome, autism, Parkinson's disease, depression, anxiety or anxiety disorder, Asperger syndrome, glucose intolerance, diabetes, reactive hypoglycemia, metabolic syndrome, low cortisol, hypothalamus-pituitary-adrenal dysfunction, myasthenia gravis syndrome, osteoporosis, autoimmune polyendocrine syndrome, chronic fatigue syndrome (CFS), central sensitivity syndrome, angina, syndrome X, chronic neck pain syndrome, chronic neuromuscular pain, osteoarthritis, muscle tension headache, chronic headache, cluster headache, temporalis tendonitis, sinusitis, atypical facial pain, trigeminal neuralgia, facial and neck pain syndrome, temperomandibular joint syndrome, idiopathic chronic low back pain, endometriosis, painful abdominal adhesions, chronic abdominal pain syndrome, coccydynia, pelvic floor myalgia (levator ani spasm), polymyositis, postherpetic neuralgia, polyradiculoneuropathies, mononeuritis multiplex, reflex sympathetic dystrophy, neuropathic pain, vulvar vestibulitis, vulvodynia, chronic regional pain syndrome, osteoarthritis, fibrositis, chronic visceral pain syndrome, female urethral syndrome, painful diverticular disease, functional dyspepsia, nonulcer dyspepsia, non-erosive esophageal reflux disease, acid-sensitive esophagus, interstitial cystitis, chronic pelvic pain syndrome, chronic urethral syndrome, chronic prostatitis, primary dysmenorrheal, dyspareunia, premenstrual syndrome (PMS), vulvodynia, ovarian remnant syndrome, ovulatory pain, pelvic congestion syndrome, myofasical pain syndrome, fibromyalgia polymyalgia rheumatica, Reiter's syndrome (reactive arthritis), rheumatoid arthritis, spondyloarthropathy, functional somatic syndromes, chronic regional pain syndromes, post-polio syndrome, functional somatic syndrome, rhinitis, asthma, multiple chemical sensitivity syndrome, reactive airway dysfunction syndrome, dysnomia, sick building syndrome, asthma, idiopathic pulmonary fibrosis, idiopathic pulmonary hypertension, dysphagia, gastroparesis, functional diarrhea, chronic constipation, defecation dysfunction, dysuria, atonic bladder, neurogenic bladder, irritable bowel syndrome (IBS), ileus, chronic idiopathic pseudoobstruction, Ogilvie's syndrome, restless leg syndrome, immune dysfunction syndrome, multiple sclerosis (MS), eczema, psoriasis, atopic dermatitis, dermatitis, Crohn's disease, ulcerative colitis, ulcerative proctitis, pouchitis, nonspecific ulcerative colitis, inflammatory bowel disease (IBD), celiac disease, diversion colitis, collagenous colitis, lymphocytic colitis, blind loop syndrome, nonalcoholic steatohepatitis (NASH), fatty liver, chronic liver disease, cirrhosis, spontaneous bacterial peritonitis, postoperative ileus, systemic lupus erythematosis, mixed connective tissue disorder, undifferentiated connective tissue disorder, Raynaud's phenomenon, Kawasaki syndrome, polymyositis, dermatomyositis, myositis, multiple autoimmune syndrome, Sjögren's syndrome, lichen planus, idiopathic uveitis, gingivitis, stomatitis, otitis, necrotizing enterocolitis, intensive care unit (ICU) multiple organ failure, primary biliary cirrhosis, idiopathic myelofibrosis, polyarteritis nodosa, eosinophilic pleural effusion, eosinophilic gastroenteritis, eosinophilic esophagitis, graft vs. host disease, Grave's disease, idiopathic thyroid failure, Hashimoto's thyroiditis, autoimmune hepatitis, pancreatitis, CREST syndrome, autoimmune cholangitis, ankylosing spondylitis, atopic dermatitis, vitiligo, scleroderma, autoimmune ear disease, polyangiitis overlap syndrome, primary sclerosing cholangitis, Gulf War syndrome, myalgic encephalomyelitis, food sensitivity, dysregulation spectrum syndrome, post-traumatic stress disorder (PTSD), benign tumor, malignant tumor, cancer and combinations thereof.

In various embodiments, the active pharmaceutical ingredient in the drug delivery device of the present disclosure can be selected from the group consisting of atazanavir, didanosine, efavirenz, emtricitabine, lamivudine, lopinavir, nevirapine, raltegravir, ritonavir, saquinavir, stavudine, tenofovir, tenofovir disoproxil fumarate, zidovudine, acyclovir, famciclovir, valcyclovir, morphine, buprenorphine, estrogen, progestin, progesterone, cyclosporine, a calcineurin inhibitor, prostaglandin, a beta-blocker, gentamycin, corticosteroid, a fluoroquinolone, insulin, an antineoplastic drug, anti-nausea drug, a corticosteroid, an antibiotic, morphine buprenorphine, a VEGF inhibitor, GnRH agonists, GnRH antagonists, GLP-1 agonists, opioid agonists, opioid antagonists, somatostatin analogs, integrase strand transfer inhibitors, integrase inhibitors, non-nucleoside reverse-transcriptase inhibitors, helicase-primase inhibitor, human parathyroid hormone, and combinations thereof.

In some embodiments, combinations of different co-prescribed drugs can be used in a drug delivery systems to treat a same disease. Examples of combinations of drugs that can be used to treat a same disease include etonogestrel and ethinyl estradiol for contraception, acyclovir and pritelivir to treat and prevent genital herpes, dolutegravir and rilpivirine for HIV pre-exposure prophylaxis, cabotegravir and rilpivirine for HIV pre-exposure prophylaxis, tenofovir disoproxil fumarate and emtricitabine for HIV pre-exposure prophylaxis, tenofovir disoproxil fumarate, emtricitabine and maraviroc for HIV pre-exposure prophylaxis, leuprolide and estradiol for endometriosis and fibroids. In the case of leuprolide and estradiol combination, estradiol is used as an add-back therapy to overcome the leuprolide-associated reduction in bone mass density.

In some embodiments, a drug delivery system can be loaded with a same or different drugs to provide a multipurpose approach for treating one or multiple diseases. Examples of same drugs used to treat multiple diseases include leuprolide for endometriosis and contraception, acyclovir for the treatment of genital herpes and for the prevention of transmission of genital herpes, and pritelivir for the treatment of genital herpes and for the prevention of transmission of genital herpes.

In some other embodiments, a drug delivery system can be loaded with different drugs to treat different diseases. Example of drug combinations to treat different diseases include ttonogestrel, ethinyl estradiol, and pritelivir for combined contraception and to treat/prevent transmission of genital herpes, dolutegravir, rilpivirine, and pritelivir for HIV pre-exposure prophylaxis and to treat/prevent transmission of genital herpes, leuprolide and etonogestrel and ethinyl estradiol for contraception and endometriosis, exenatide and pritelivir for diabetes and herpes, leuprolide and estradiol for endometriosis and fibroids, and octreotide and pritelivir for the treatment of acromegaly and to treat/prevent transmission of genital herpes.

For example, in some embodiments, drug delivery systems herein described and in particular the IVR can be used for protection from multiple infections such as Human Immunodeficiency Virus (HIV) and Herpes Simplex Virus (HSV) infections, and/or for protection from unintended pregnancy through the development of pod-based intravaginal ring (IVR) formulations.

In particular, some embodiments, the drug delivery systems herein described can be used for HIV Pre-Exposure Prophylaxis (PrEP). In particular in some of these embodiments, possible drugs that can be delivered with delivery devices of the disclosure comprise Tenofovir Alafenamide (TAF) Tenofovir disoproxil fumarate (TDF), Emtricitabine (FTC), dapivirine. In particular, the drug delivery systems of the present disclosure can be used to deliver an IVR formulation of TDF-TFC for HIV PrEP, also or preferably in combination with contraception, as anti-HSV.

In some embodiments, the drug delivery systems herein described can be used for treatment or prophylaxis of Herpes Simplex Virus (HSV). In particular in some of these embodiments, possible drugs that can be delivered with delivery devices of the disclosure comprise valacyclovir, Pritelivir and additional drugs identifiable by a skilled person. In those embodiments it is expected that an effective anti-herpes IVR could lead not only to symptomatic relief and fewer recurrences in HSV+ women but could also provide pre-exposure prophylaxis for both HSV and HIV.

In some embodiments, the drug delivery systems herein described can be used for treatment or prophylaxis of Human Papilloma Virus (HPV). In particular in some of these embodiments, possible drugs that can be delivered with delivery devices of the disclosure comprise Ranpirase.

In some embodiments, the drug delivery systems herein described can be used for protection from unintended pregnancy. It is expected that an. The hormonal contraceptives, Etonogestrel (ETG) and Ethinyl Estradiol (EE) are used as combination therapy and commercially sold as an IVR-NuvaRing®. The IVR described herein that can deliver these two hormones at rates comparable to the NuvaRing® and can be combined in a single IVR.

In some embodiments, the three applications described above can be combined and the different drugs co-prescribed as a multipurpose prevention technology (MPT) approach to contraception and viral pre-exposure prophylaxis. The following sections describe how an IVR-based MPT approach can be advantageously based on an intravaginal ring, as well as advantages in combining HIV and HSV PrEP with contraception. Certain advantages of the specific drugs referred to above is also described, though other drugs may also be used.

In some embodiments the drug delivery system can be provided in the form of a kit of parts comprising at least two of one or more drug pods, in particular comprising the drug cores, drug cores, and blank carrier herein described in any possible configuration as will be understood by a skilled person.

In a particular embodiment, kits may include single or multiple doses of one or more active ingredients, each packaged or formulated individually in one or more drug cores possibly already inside a drug pod, or single or multiple doses of two or more active ingredients packaged or formulated in combination within one or more drug core possibly already included in inside a drug pod herein described. Thus, one or more first active ingredients may be present in a first drug pod, and one or more second active ingredients in a second drug pod. The drug pod, blank carrier and/or or containers are placed within a package in a ready to use configuration or in configurations where the parts are comprised separately and the configuration used is selected by the individual, pharmacist and/or physician. In some embodiments the package may optionally include administration or dosage instructions in the form of a label on the package or in the form of an insert included in the packaging of the kit.

In some embodiments, the delivery system herein described can be placed by the user in body cavities (e.g. in a human vagina or rectum). In some embodiments, the delivery system herein described can be surgically implanted at a target location, in or near the site of action (e.g. in vagina, uterus pelvis, rectum, eye ear, nose, sinus prostate and bladder) and/or at a site allowing system administration (e.g. subcutaneously, intramuscularly or intraperitoneally) for example in applications when devices are to give sustained systemic levels and avoid premature metabolism.

The drug pod combination, amounts of API, related drug core formulations including number and concentration of excipients, configuration of drug pod and relate materials and dimensions depend on the method of administration, the effective agent used, the polymers used, the desired release rate. In particular, the release rates and release duration depend on a variety of factors in addition to the above such as the disease state being treated, the age and condition of the patient, the route of administration as well as other factors which would be apparent to a skilled person.

In particular as with any API and pharmaceuticals, it will be understood that the total daily usage of one or more pharmaceutical compositions included in the drug pods of the delivery system herein described is typically decided by a patient's attending physician within the scope of sound medical judgment. The specific therapeutically effective or prophylactically effective dose level for any particular patient will depend upon a variety of factors including the disorder being treated and the severity of the disorder; activity of the specific compound employed; the specific composition employed; the age, body weight, general health, sex and diet of the patient; the time administration, route of administration, and rate of excretion of the specific compound employed; the duration of the treatment; drugs used in combination or coincidental with the specific compound employed; and other factors known to those of ordinary skill in the medical arts.

### EXAMPLES

The drug delivery devices, related components, materials, compositions, methods system herein described are further illustrated in the following examples, which are provided by way of illustration and are not intended to be limiting.

In particular, the following examples illustrate properties exemplary drug delivery devices and related methods and systems. A person skilled in the art will appreciate the applicability and the necessary modifications to adapt the features described in detail in the present section, to additional associative polymers, compositions, methods and systems according to embodiments of the present disclosure.

### Example 1: Drug Delivery Efficacy of a Prior Art IVR System without Removable Pods

The efficacy of an IVR releasing TDF-FTC was demonstrated in a repeated challenge primate transmission study.

The trial consisted of an open-label, crossover design where six participants sequentially used a TDF pod-IVR for seven days followed by a TDF-FTC pod-IVR, with a washout period of at least 14 days between each treatment period. Women who were clinically deemed eligible returned to the clinic for IVR insertion visit (Visit 1, TDF IVR; Visit 5, TDF-FTC IVR) after cessation of menses. Women returned for Visit 2 (TDF IVR) or Visit 6 (TDF-FTC IVR) on Day 2 (±1 d) after IVR insertion and for Visit 3 (TDF IVR) or Visit 7 (TDF-FTC IVR) on Day 7 (±1 d) when the IVRs were removed. At Visit 3/7, vaginal biopsies were obtained, and the IVRs were collected. Tissue samples were ultimately reported as ng/mg or fmol/mg, respectively, following normalization to net biopsy or Dacron swab weight.

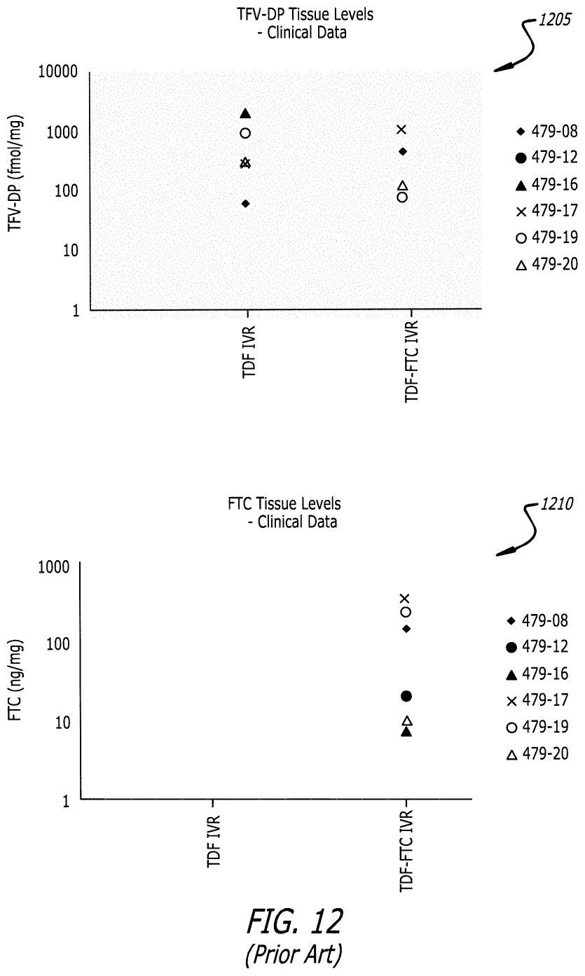

The related results are illustrated in FIG. 12 which illustrates vaginal biopsy homogenate TFV-DP levels at 7 days (1205) and vaginal biopsy homogenate FTC levels (1210). None of the IVRs showed any significant safety concerns assessed by AEs, colposcopy, epithelial evaluation, vaginal microbiome, and histopathology. As can be noted from FIG. 12, tissue levels after 7 days were high enough for PrEP action.

Although FIG. 12 refers to a previous prototype ring as illustrated in FIG. 13, similar or better results may be expected with the new style ring described herein as shown by the data in Examples 4 to 6 below.

### Example 2: Drug Delivery System for Contraception and Treatment of Genital Herpes with a Prior Art IVR without Removable Pods

The following sections describe preliminary data for pod-in-ring prior art IVRs such as the one illustrated in FIG. 13 and in particular: a TDF-FTC ring for HIV PrEP; an ETG-EE ring for contraception; and an ACV ring for genital herpes.

For the preliminary data for a TDF-FTC pod-IVR, an IVR releasing the APIs of Truvada® (TDF-FTC) was evaluated in clinical safety and PK study in a 7-day clinical trial under Auritec-sponsored IND No. 123099. The results are illustrate in FIG. 12, as discussed above in the present disclosure.

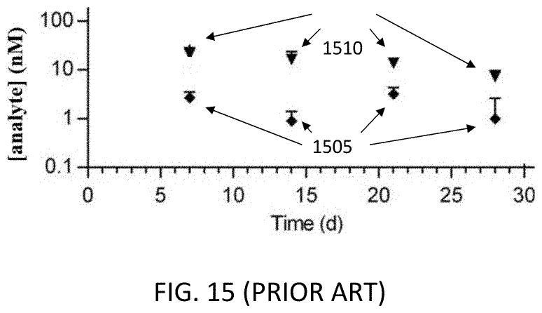

FIG. 15 illustrates a graph of ETG-EE release: ETG (1510); EE (1505). The pod-in-rings for this example delivered etonogestrel (ETG) and estradiol (EE). The silicone pod IVRs contained two pods of EE at 16 mg API per pod (32 mg drug per ring) and ETG at 10 mg API per pod (20 mg drug per ring). Median ETG CVL levels (13±6.9 nM) were 8 times higher than the corresponding estradiol (E2) levels (1.6±1.2 nM), in agreement with the relative daily release rates of ETG and EE from the NuvaRing® (120 μg day⁻¹ and 15 μg day⁻¹, respectively). Plasma levels were not measured, the preliminary results should imply a match for in vivo and in vitro release compared to Nuvaring®.

Similar drug release rates and corresponding delivered concentrations are expected to result from the use of an IVR delivery system with removable pod according to the present disclosure as shown by the data reported in Examples 4 to 6 below.

### Example 3: Clinical Study of Anti-HSV Activity in Blood and Cervicovaginal Lavage (CVL) with a Prior Art IVR System without Removable Pods

A clinical study was performed with a prior art IVR without removable pods such as the one described in FIG. 13.

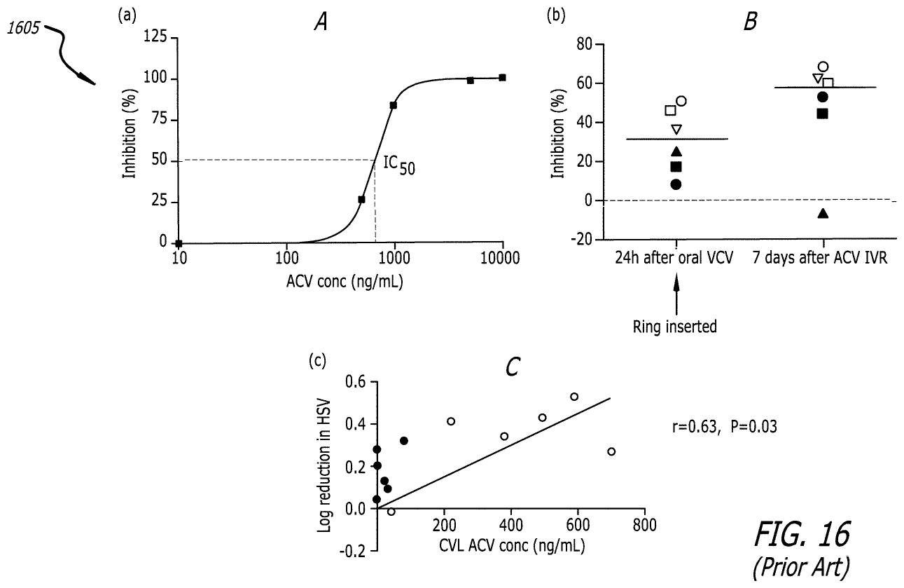

Six HSV+ women recurrent genital HSV switched their daily oral valacyclovir suppression to an acyclovir (ACV) IVR for 7 days (n=3) or 14 days (n=3), under IND (No 108,536) for a staged clinical trial of a ring releasing acyclovir for the treatment and prevention of genital herpes. Tolerability and PK were evaluated and antiviral activity of cervicovaginal lavage (CVL) samples was correlated with drug levels in the samples. This first-in-human study in women of an ACV IVR demonstrates that genital tract levels of ACV that are similar to oral valacyclovir can be delivered without systemic absorption. The ring was well tolerated with no colposcopic findings or significant changes in inflammatory cytokine or chemokine concentrations in CVL. The results are illustrated in FIG. 16.

In FIG. 16, panel A (1605), the percentage inhibition of HSV infection was determined in the presence of increasing concentrations of acyclovir (ACV). In panel B (1610) the percentage inhibition of HSV infection was determined in the presence of the indicated CVL sample or control fluid. Results are means from duplicate wells and each participant is represented by a different symbol (1615); the horizontal lines (1620) indicate the medians for the groups. In panel C (1625), the relationship between drug levels and percentage inhibition was determined by the Spearman correlation coefficient. Filled circles (1630) represent CVL samples obtained 24 h after oral VCV dosing, and open circles (1635) are CVL samples obtained 7 days after ring insertion.

A similar effectiveness of the drug release is expected from the use of an IVR delivery system with removable pod according to the present disclosure as also shown by the results shown in Example 6.

### Example 4: In Vitro Study of Pritelivir Removable Pods

Genital herpes is a significant medical problem in the United States with approximately 50 million people affected. Pritelivir is a very promising new drug but has shown significant toxicity when delivered systemically. For this reason, local delivery of pritelivir as intravaginal ring has assumed significant medical and financial importance.

In this example, compressed pods of Pritelivir were prepared using standard laboratory-scale tableting methods. The drug substance was weighed and placed in a hydraulically actuated manual pellet press to produce cylindrical tablets. To measure in vitro release, the drug pods were placed in 250 mL jars containing a 50/50 mixture of water and isopropanol. Jars were placed into an orbital shaker at 37° C. and 60 RPM.

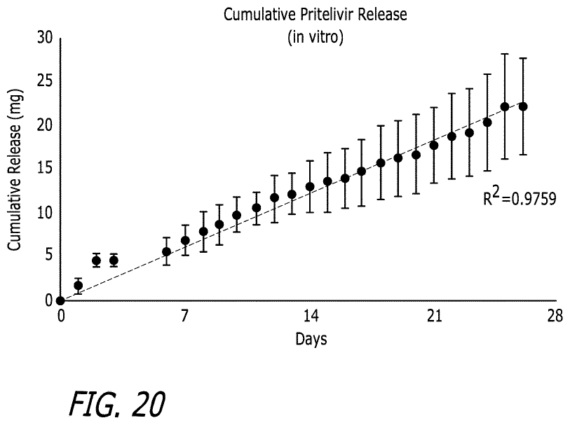

The dissolution media was sampled daily and changed in order to preserve sink conditions. Samples were analyzed for drug concentration using an HPLC method. The dissolution results are presented in FIG. 20 in which the cumulative release of pritelivir pods are recorded for 28 days.

### Example 5: In Vitro Study of Octreotide Removable Pods

Acromegaly is a serious condition which is currently only treated by injectable drugs. For this reason, an intravaginal ring which could avoid injections would be of commercial and medical value.

In this example, compressed pods of octreotide were prepared using standard laboratory-scale tableting methods. The drug substance was weighed and placed in a hydraulically actuated manual pellet press to produce cylindrical tablets. To measure in vitro release, the drug pods were placed in 250 mL jars containing a vaginal fluid simulant (VFS). Jars were placed into an orbital shaker at 37° C. and 60 RPM.

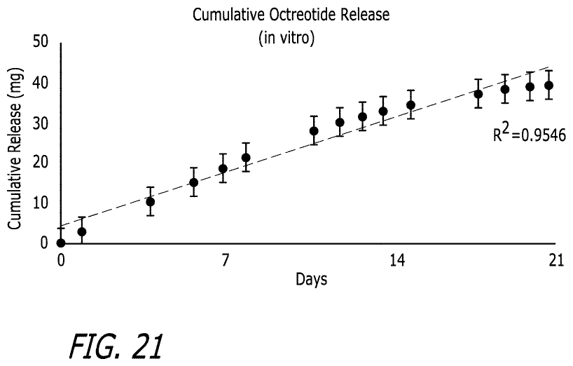

Samples were analyzed for drug concentration using an HPLC method. The dissolution results are presented in FIG. 21 in which the cumulative release of octreotide pods are recorded for 21 days.

### Example 6: In Vivo Study of an Intravaginal Ring with Octreotide and Pritelivir Removable Pods in Sheep

IVR delivery is a platform that can deliver peptides at clinically relevant levels. A sheep study was performed to deliver octreotide intravaginally in combination with pritelivir, a drug that is in clinical trials for the treatment of genital herpes. Both, octreotide and pritelivir, were formulated as removable pods herein described.

In particular a study was conducted in sheep to measure the safety and pharmacokinetics of an intravaginal ring containing removable pods of octreotide, pritelivir and carboxymethylcellulose (placebo) pods. Cervico-vaginal lavage (CVL) samples were collected at Days 1, 3, 7, 14, 21 and 28 post-IVR insertion. The study was conducted in 9 sheep with 3 groups (3 sheep per group), each group being given different dosage of octreotide and pritelivir:

Group 1: low-dose octreotide; high-dose pritelivir;

Group 2: mid-dose octreotide; mid-dose pritelivir;

Group 3: high-dose octreotide; low-dose pritelivir

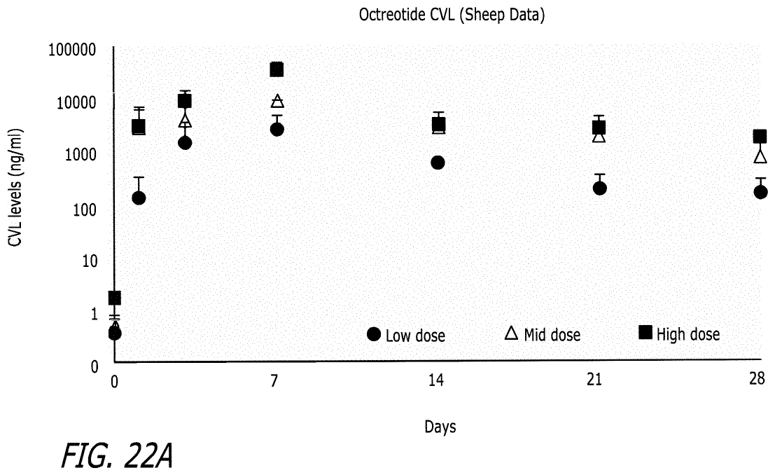

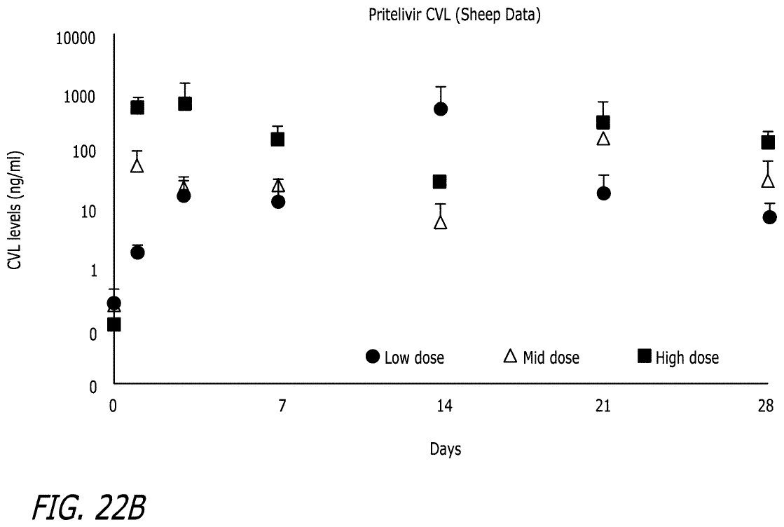

The results are shown in FIGS. 22 A-B. FIG. 22A illustrates levels of octreotide in the sheep cervicovaginal lavage. Low dose=1 pod of octreotide; Mid dose=2 pods of octreotide; High dose=4 pods of octreotide. FIG. 22B illustrates levels of pritelivir in the sheep cervicovaginal lavage. Low dose=1 pod of pritelivir; Mid dose=3 pods of pritelivir; High dose=9 pods of pritelivir.

This example shows that drugs of medical benefit of different classes can be delivered together. Pritelivir is a small molecule, while octreotide is a large molecule (peptide). Pritelivir is insoluble in water, while octreotide is very soluble.

Furthermore, the pritelivir pod is approvable by itself by showing safety and efficacy in clinical trials of genital herpes. The octreotide pod is approvable by proof of safety and efficacy in acromegaly. Should a patient with genital herpes develop acromegaly, the patient could be prescribed both pods individually without the requirement for a clinical trial.

In summary provided herein are a drug delivery device with removable pods is described and related pods methods and systems. The drug delivery device comprises a plurality of receptacles, each receptacle configured to accept a corresponding drug pod, wherein each receptacle comprises: a recess configured to accept a protrusion in the corresponding drug pod, or a protrusion configured to insert in a recess in the corresponding drug pod. The corresponding drug pod comprises a shell, made of an impermeable polymer, having a first base comprising an opening configured to allow delivery of a drug, a second base, a lateral surface, a protrusion attached to, and extending away from, the lateral surface, or a recess in the lateral surface.

In particular in several embodiments a drug delivery system herein described comprises multiple openings possibly on one side, each opening accepting a receptacle. Each receptacle can accept a shell containing a drug core. Each drug core includes an active pharmaceutical ingredient and an excipient. The shell is made of an impermeable polymer, allowing the drug release through an opening sealed with a semi-permeable polymer. The semi-permeable polymer which allows the active compound to be released at a controllable rate can be inserted and cured within the shell prior to insertion of the drug core. The system allows for the controlled release of multiple drugs in an intravaginal environment.

The examples set forth above are provided to give those of ordinary skill in the art a complete disclosure and description of how to make and use the embodiments of the materials, compositions, systems and methods of the disclosure, and are not intended to limit the scope of what the inventors regard as their disclosure. Those skilled in the art will recognize how to adapt the features of the exemplified methods and arrangements to additional tunable surfactants, and related compositions, methods and systems, in according to various embodiments and scope of the claims.

All patents and publications mentioned in the specification are indicative of the levels of skill of those skilled in the art to which the disclosure pertains. The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the devices, pods and related methods and systems of the present disclosure and it will be apparent to one skilled in the art that the disclosure can be carried out using a large number of variations of the devices, device components, methods steps set forth in the present description. As will be obvious to one of skill in the art, methods and devices useful for the present methods may include a large number of optional composition and processing elements and steps.

In particular, it will be understood that various modifications may be made without departing from the spirit and scope of the present disclosure. Accordingly, other embodiments are within the scope of the following claims.

1. A removable drug pod for a drug delivery device, said removable drug pod comprising:
a shell, made of an impermeable polymer, having a first base comprising an opening configured to allow delivery of a drug, a second base, a lateral surface,
a protrusion attached to, and extending away from, the lateral surface, or
a recess in the lateral surface,
the protrusion or recess being configured to allow self-anchoring of the protrusion or recess and retainment of the removable pod within the drug delivery device;
and optionally
(i) a semi-permeable polymer layer and/or permeable polymer on the first base, the semi-permeable and/or permeable polymer layer covering the opening in the first base, and/or
(ii) a drug core within the shell.

2. The removable drug pod of claim 1, wherein the opening has a circular cross section and the protrusion is cylindrical.

3. The removable drug pod of claim 1, wherein the drug core comprises a pill coated with a semi-permeable polymer.

4. The removable drug pod of claim 1, wherein the drug core comprises an active pharmaceutical ingredient and an excipient.

5. The removable drug pod of claim 1, wherein the impermeable polymer is silicone and the second base is a silicone adhesive.

6. The removable drug pod of claim 1, wherein the protrusion is annular.

7. The removable drug pod of claim 1, wherein the opening is sealed with a semi-permeable polymer.

8. The removable drug pod of claim 1, wherein the semi-permeable polymer on the first base is poly(vinyl alcohol).

9. The removable drug pod of claim 7, wherein the semi-permeable polymer sealing the opening is poly(vinyl alcohol).

10. The removable drug pod of claim 1, wherein the protrusion or recess is configured to snap-fit within the drug delivery device.

11. A blank carrier ring device comprising a ring, the ring having a plurality of cylindrical openings, each opening configured to removably accept a corresponding drug pod,
wherein the corresponding drug prod is the removable drug pod according to claim 1 and
wherein each opening comprises:
a recess configured to accept a protrusion in the corresponding drug pod, or
a protrusion configured to insert in a recess in the corresponding drug pod.

12. The blank carrier ring device of claim 11, wherein the ring is made of silicone.

13. A drug delivery system comprising:
the blank carrier ring device according to claim 11, comprising a ring, the ring having a plurality of receptacles, each receptacle configured to accept a corresponding drug pod, wherein each receptacle comprises:
a recess configured to accept a protrusion in the corresponding drug pod, or
a protrusion configured to insert in a recess in the corresponding drug pod; and
at least one said drug pod according to any one of claims 1 to 10, configured to be inserted in a receptacle of the plurality of receptacles, the at least one drug pod comprising:
a shell, made of an impermeable polymer, having a first base comprising an opening configured to allow delivery of a drug, a second base, a lateral surface, a protrusion attached to, and extending away from, the lateral surface, and optionally a semi-permeable polymer layer on the first base, the semi-permeable polymer layer covering the opening in the first base, and
a drug core within the shell.

14. The drug delivery system of claim 13, wherein the at least one drug pod comprises a plurality of drug pods, each comprising one drug core.

15. The drug delivery system of claim 13, wherein:
the opening has a circular cross section,
each drug core comprises an active pharmaceutical ingredient and an excipient, and
the opening is sealed with a semi-permeable polymer.

16. The drug delivery system of claim 13, wherein the protrusion is cylindrical or annular.

17. The drug delivery system of claim 13, wherein the impermeable polymer is silicone, the second base is a silicone adhesive, and the semi-permeable polymer is poly(vinyl alcohol).

18. The drug delivery system of claim 13, wherein each pill comprises a different active pharmaceutical ingredient than other pills.

19. The drug delivery system of claim 13, wherein the plurality of receptacles comprises ten receptacles.

20. A method comprising:
fabricating a pill comprising an active pharmaceutical ingredient and an excipient;
inserting the pill in a shell, made of an impermeable polymer, the shell having a first base comprising an opening configured to allow delivery of a drug, a second empty base, a lateral surface, and optionally a semi-permeable polymer layer on the first base, the semi-permeable polymer layer covering the opening in the first base, the inserting being carried out through the second empty base;
sealing the second empty base with an adhesive; and
sealing the opening with a second semi-permeable polymer, thereby obtaining the removable drug pod of claim 1 comprising a sealed shell containing a pill,
wherein the shell comprises:
a protrusion attached to, and extending away from, the lateral surface, or
a recess in the lateral surface.

21. The method of claim 20, wherein the impermeable polymer is silicone, the adhesive is silicone, and the first and second semi-permeable polymer are poly(vinyl alcohol).

22. The method of claim 20, further comprising:
inserting a plurality of drug pods comprising sealed shells each containing a different pill in corresponding receptacles of a ring-shaped blank carrier device, thereby obtaining the drug delivery system of any one of claims 13 to 19 loaded carrier device.

23. The method of claim 22, wherein the loaded carrier device is configured for vaginal insertion.

24. The drug delivery system of claim 13 for use in contraception and/or treatment and/or prevention of a disease in an individual, wherein
the drug delivery system comprises plurality of drug cores within a plurality of drug pods, each drug core of the plurality of drug core within a corresponding pod of the plurality of drug pods, each drug core of the plurality of drug core and corresponding pod selected to provide the individual with one or more target active pharmaceutical ingredient at one or more effective target concentrations.

25. The drug delivery system of claim 24, wherein the disease is selected from the group consisting of vaginal disease, uterine disease, pelvic disease, rectal disease, eye disease, ear disease, sinus disease, nasal disease, prostatic disease, and bladder disease.

26. The drug delivery system of claim 24, wherein the disease is selected from the group consisting of hyperhomocysteinemia, chronic renal failure, end stage renal disease, hemodialysis, peritoneal dialysis, vascular dementia, cardiovascular disease, stroke, cerebrovascular accidents, thrombotic disorder, hypercoagulable states, venous thrombosis, deep vein thrombosis, thrombophlebitis, thromboembolic disease, ischemic stroke, restenosis after percutaneous transluminal coronary angioplasty (PTCA), preeclampsia, vasculitis, digital ischemia, multifocal osteonecrosis, retinal vein occlusion, glaucoma, miscarriage, pregnancy complication, placental abruption, transplantation, diabetic retinopathy, ischemic bowel disease, cerebral vein thrombosis, atherosclerosis, coronary artery disease, penile venous thrombosis, impotence, central venous thrombosis, peripheral artery disease, intermittent claudication, hemorrhagic colitis, radiation enteritis, radiation colitis, visceral ischemia, acute mesenteric ischemia, chronic mesenteric ischemia, hypertension, microangiopathy, macroangiopathy, recurrent leg ulcer, carotid stenosis, occlusive vascular disease, arterial aneurysm, abdominal aortic aneurysm, congestive heart failure, hepatopulmonary syndrome, high flow state associated with chronic liver disease, migraine headache, vascular headache, dizziness, lightheadedness, orthostatic intolerance, postural hypotension, postural hypotension, postural orthostatic tachycardia syndrome, idiopathic pulmonary fibrosis, pulmonary hypertension, angioedema, vaso-vagal faints, neuroleptic malignant syndrome, learning disorder, learning disability, insomnia, dementia, age associated memory impairment, attention deficit/hyperactivity disorder (ADHD), mild cognitive impairment, Alzheimer's disease, Down's syndrome, autism, Parkinson's disease, depression, anxiety or anxiety disorder, Asperger syndrome, glucose intolerance, diabetes, reactive hypoglycemia, metabolic syndrome, low cortisol, hypothalamus-pituitary-adrenal dysfunction, myasthenia gravis syndrome, osteoporosis, autoimmune polyendocrine syndrome, chronic fatigue syndrome (CFS), central sensitivity syndrome, angina, syndrome X, chronic neck pain syndrome, chronic neuromuscular pain, osteoarthritis, muscle tension headache, chronic headache, cluster headache, temporalis tendonitis, sinusitis, atypical facial pain, trigeminal neuralgia, facial and neck pain syndrome, temperomandibular joint syndrome, idiopathic chronic low back pain, endometriosis, uterine fibroids, acromegaly, neuroendocrine tumors, painful abdominal adhesions, chronic abdominal pain syndrome, coccydynia, pelvic floor myalgia (levator am spasm), polymyositis, postherpetic neuralgia, polyradiculoneuropathies, mononeuritis multiplex, reflex sympathetic dystrophy, neuropathic pain, vulvar vestibulitis, vulvodynia, chronic regional pain syndrome, osteoarthritis, fibrositis, chronic visceral pain syndrome, female urethral syndrome, painful diverticular disease, functional dyspepsia, nonulcer dyspepsia, non-erosive esophageal reflux disease, acid-sensitive esophagus, interstitial cystitis, chronic pelvic pam syndrome, chronic urethral syndrome, chronic prostatitis, pnmary dysmenorrheal, dyspareunia, premenstrual syndrome (PMS), vulvodynia, ovarian remnant syndrome, ovulatory pain, pelvic congestion syndrome, myofasical pain syndrome, fibromyalgia polymyalgia rheumatica, Reiter's syndrome (reactive arthritis), rheumatoid arthritis, spondyloarthropathy, functional somatic syndromes, chronic regional pain syndromes, post-polio syndrome, functional somatic syndrome, rhinitis, asthma, multiple chemical sensitivity syndrome, reactive airway dysfunction syndrome, dysnomia, sick building syndrome, asthma, idiopathic pulmonary fibrosis, idiopathic pulmonary hypertension, dysphagia, gastroparesis, functional diarrhea, chronic constipation, defecation dysfunction, dysuria, atonic bladder, neurogenic bladder, irritable bowel syndrome (IBS), ileus, chronic idiopathic pseudoobstruction, Ogilvie's syndrome, restless leg syndrome, immune dysfunction syndrome, multiple sclerosis (MS), eczema, psoriasis, atopic dermatitis, dermatitis, Crohn's disease, ulcerative colitis, ulcerative proctitis, pouchitis, nonspecific ulcerative colitis, inflammatory bowel disease (IBD), celiac disease, diversion colitis, collagenous colitis, lymphocytic colitis, blind loop syndrome, nonalcoholic steatohepatitis (NASH), fatty liver, chronic liver disease, cirrhosis, spontaneous bacterial peritonitis, postoperative ileus, systemic lupus erythematosis, mixed connective tissue disorder, undifferentiated connective tissue disorder, Raynaud's phenomenon, Kawasaki syndrome, polymyositis, dermatomyositis, myositis, multiple autoimmune syndrome, Sj6gren's syndrome, lichen planus, idiopathic uveitis, gingivitis, stomatitis, otitis, necrotizing enterocolitis, intensive care unit (ICU) multiple organ failure, primary biliary cirrhosis, idiopathic myelofibrosis, polyarteritis nodosa, eosinophilic pleural effusion, eosinophilic gastroenteritis, eosinophilic esophagitis, graft vs. host disease, Grave's disease, idiopathic thyroid failure, Hashimoto's thyroiditis, autoimmune hepatitis, pancreatitis, CREST syndrome, autoimmune cholangitis, ankylosing spondylitis, atopic dermatitis, vitiligo, scleroderma, autoimmune ear disease, polyangiitis overlap syndrome, primary sclerosing cholangitis, Gulf War syndrome, myalgic encephalomyelitis, food sensitivity, dysregulation spectrum syndrome, post traumatic stress disorder (PTSD), benign tumor, malignant tumor, cancer and combinations thereof.

27. The drug delivery system of claim 24, wherein the active pharmaceutical ingredient in the drug delivery system is selected from the group consisting of atazanavir, didanosine, efavirenz, emtricitabine, lamivudine, lopinavir, nevirapine, raltegravir, ritonavir, saquinavir, stavudine, tenofovir, tenofovir disoproxil fumarate, zidovudine, acyclovir, famciclovir, valcyclovir, morphine, buprenorphine, estrogen, progestin, progesterone, cyclosporine, a calcineurin inhibitor, prostaglandin, a beta-blocker, gentamycin, corticosteroid, a fluoroquinolone, insulin, an antineoplastic drug, anti-nausea drug, a corticosteroid, an antibiotic, morphine buprenorphine, a VEGF inhibitor, GnRH agonists, GnRH antagonists, GLP-1 agonists, opioid agonists, opioid antagonists, somatostatin analogs, integrase strand transfer inhibitors, integrase inhibitors, non-nucleoside reverse-transcriptase inhibitors, helicase-primase inhibitor, human parathyroid hormone, and combinations thereof.

28. The drug delivery system of claim 21, wherein the drug delivery device is adapted for a route of administration selected from the group consisting of: sub-dermal, sub-cutaneous, systemic, local, epidural, intra-lesional, intra-tumor, intra-punctal and combinations thereof.
